(19) United States
(12) Patent Application Publication
(10) Pub. No.: US 2009/0023637 A1
(43) Pub. Date: Jan. 22, 2009

(54) Methods for detecting and treating interstitial cystitis

(76) Inventor: C. Lowell Parsons, Henderson, NV (US)

Correspondence Address: MANDEL & ADRIANO, 572 EAST GREEN STREET, SUITE 203, PASADENA, CA, 91101, US

(21) Appl. No.: 12/002,056

(22) Filed: Dec. 14, 2007

Related U.S. Application Data
(63) Continuation-in-part of application No. PCT/US2006/033544, filed on Aug. 30, 2006.
(60) Provisional application No. 60/875,025, filed on Dec. 15, 2006.

(51) Int. Cl. A61K 38/17; G01N 33/68; A61P 13/10

(52) U.S. Cl. 514/8; 436/87

(57) ABSTRACT

The invention provides a method for diagnosing and inhibiting conditions associated with reduced amounts of THP and/or reduced amounts of total carbohydrate content in the THP, such as Interstitial Cystitis and its symptoms, and damaged mucin layers in a subject by administering an effective amount of a Tamm-Horsfall protein to the subject. The invention also provides a method for diagnosing Interstitial Cystitis in a subject by quantitatively determining in a sample, the level of THP, the amount of sialylation of Tamm-Horsfall protein and/or the total carbohydrate content in THP. A decrease in the amounts of THP, sialylation of Tamm-Horsfall protein and/or carbohydrate content in THP is indicative of Interstitial Cystitis.

This application is a continuation-in-part of (under 35 U.S.C. §111(a)), and claims the priority to (under 35 U.S.C. §§120 and 365(c)), PCT application PCT/US2006/033544, with international filing date Aug. 30, 2006, which claims the priority of U.S. Ser. No. 60/172,632 filed on Aug. 30, 2005 and this application also claims the priority of U.S. Ser. No. 60/875,025 filed on Dec. 15, 2006, the entirety of all of which are hereby incorporated by reference into this application.

Throughout this application various publications are referenced. The disclosures of these publications in their entireties are hereby incorporated by reference into this application in order to more fully describe the state of the art to which this invention pertains.

### FIELD OF THE INVENTION

The invention relates to methods for diagnosing, inhibiting and monitoring the course of Interstitial Cystitis. The invention also provides pharmaceutical compositions comprising sialylated Tamm-Horsfall protein, for treating Interstitial Cystitis (IC).

### BACKGROUND OF THE INVENTION

Mucus is critical in regulating epithelial permeability of the bladder, particularly to low molecular weight solutes.^{10-12, 25, 26} Additional studies have demonstrated that Interstitial Cystitis (IC), also known as Interstitial Cystitis/Bladder Pain Syndrome (IC/BPS), patients have significant impairment of epithelial permeability regulation¹³⁻¹⁶ that leads to a movement of small cations (potassium) diffusing into the membrane and interstitium of the bladder^{1-3, 18}. This diffusion initiates a cascade of nerve depolarization, muscle depolarization, and generation of symptoms of urgency, frequency, pain, and incontinence^{1-3, 8, 17, 18, 27, 28}.

Another series of experiments has shown that urine contains toxic, low molecular weight, cations that are capable of interacting with mucus and injuring its ability to regulate permeability^{4, 23, 29}. We hypothesized that these cations do not injure normal urothelium because urine contains protective substances that have affinity for these cations, interfering with their capacity to injure the urinary mucosa. This, we believe, takes place in the fluid phase of urine so that the interaction with the outer layer of mucus is prevented. In effect, the combination of urinary cations and anions is in a homeostatic balance so that toxic urinary components are less likely, if at all, able to injure the urothelium. Collectively, these interactions efficiently sequester potential toxins in urine during the storage phase in the bladder.

The experiments herein show that a molecule that acts as a critical protective factor is the highly anionic Tamm-Horsfall protein (“THP”). Synthesized in the thick ascending limb of Henie's loop in the kidney, and known as uromodulin when isolated from urine during pregnancy,⁴¹ THP is the most abundant protein in human urine.⁶ THP is a highly glycosylated and highly conserved urinary protein that is present in the urine of all vertebrates.^{5, 6} It is approximately thirty percent sugar by weight due to the presence of eight potential N-glycosylation sites that are glycosylated with various di-, tri-, and tetra-antennary N-glycans.^{42, 43} It has been investigated for a potential role in immunodefense and has been suggested to prevent urinary infection⁴⁴ and to play a role in the formation of calcium oxalate stones.⁴⁵ A number of investigators have explored its possible role in the urinary tract, but none have identified a function that would account for its ubiquity across vertebrate species.

In the past, IC/BPS was regarded as a rare disease whose symptoms and progression were difficult or impossible to control. More recent evidence has shown that IC/BPS is a relatively common disorder in both women and men, and that most cases can be treated successfully.

In the present invention, methods of diagnosing disorders of the lower urinary tract, and specifically, for detecting Interstitial Cystitis, and methods for reducing the symptoms of Interstitial Cystitis in vivo, by increasing levels of THP (for example sialylated THP), are described.

### SUMMARY OF THE INVENTION

The present invention provides methods for inhibiting Interstitial Cystitis in a subject. The method comprises administration of an effective amount of a Tamm-Horsfall protein to the subject, in a pharmaceutically acceptable carrier, so as to inhibit Interstitial Cystitis in the subject.

Further, the present invention provides a method for reducing symptoms of IC/BPS in a subject. The method comprises administration of an effective amount of Tamm-Horsfall protein to the subject in a pharmaceutically acceptable carrier.

The invention also provides a method for repairing a mucin layer of the bladder in a subject by increasing the levels of Tamm-Horsfall protein in a subject. The levels of Tamm-Horsfall protein are increased in a subject by administering an effective amount of Tamm-Horsfall protein to the subject in a pharmaceutically acceptable carrier.

Also provided by the invention is a method for treating a disease associated with decreased levels of Tamm-Horsfall protein. The method comprises increasing the levels of Tamm-Horsfall protein in a subject by administering an effective amount of Tamm-Horsfall protein in a pharmaceutically acceptable carrier, so as to treat the disease associated with reduced levels of Tamm-Horsfall protein.

Further provided in this invention is a method for diagnosing IC/BPS in a subject. The method comprises quantitatively determining in the urine from the subject, the levels of Tamm-Horsfall protein and comparing the amount of Tamm-Horsfall protein so determined to the amount in a sample from a normal subject. The decrease in the amount of Tamm-Horsfall protein in the subject compared to the normal subject is indicative of IC/BPS.

Also provided in this invention is a method for diagnosing Interstitial Cystitis/Bladder Pain Syndrome in a subject. The method comprises quantitatively determining in the urine from the subject, the amount of sialylation of Tamm-Horsfall protein and comparing the amount of sialylation Tamm-Horsfall protein so determined to the amount in a sample from a normal subject. The change in the amount of sialylation of Tamm-Horsfall protein in the subject compared to the normal subject is indicative of Interstitial Cystitis. For example, a decrease in the amount of sialylation of Tamm-Horsfall protein in the subject compared to the normal subject is indicative of Interstitial Cystitis/Bladder Pain Syndrome. In one embodiment, the sialic acid content of THP is lower in IC/BPS patients compared to the sialic acid content of THP in normal subjects. In another embodiment, the sialic acid content of urine, normalized to the urinary creatinine concentration, is lower in IC/BPS patients compared to control subjects.

Further provided in this invention is a method for diagnosing IC/BPS in a subject. The method comprises quantitatively determining in the urine from the subject, the amount of carbohydrate in Tamm-Horsfall protein in a sample from a subject and comparing the amount of carbohydrate in Tamm-Horsfall protein so determined to the amount in a sample from a normal subject. The decrease in the amount of carbohydrate in Tamm-Horsfall protein in the subject compared to a control subject is indicative of IC/BPS.

The invention also provides a method for monitoring the course of Interstitial Cystitis in a subject which comprises quantitatively determining in a first sample of a urine from the subject the levels of Tamm-Horsfall protein and comparing the amount so determined with the amount present in a second sample from the subject, such samples being taken at different points in time, a difference in the levels of Tamm-Horsfall protein (being produced by the IC (IC/BPS) patient) determined being indicative of the course of Interstitial Cystitis/Bladder Pain Syndrome.

The invention further provides a method for screening for agents that modulate (for example, increase) the production of Tamm-Horsfall protein. The method comprises contacting the Tamm-Horsfall genes in THP-positive cells with a candidate molecule of interest and then determining whether the contact results in increased Tamm-Horsfall production. An increase in Tamm-Horsfall production is indicative that the molecule modulates (for example, increase) production of Tamm-Horsfall genes.

Also provided in this invention is a method for screening for agents that modulate (for example, increase) the production of Tamm-Horsfall protein comprising contacting Tamm-Horsfall protein in THP-positive cells with a candidate molecule of interest and determining whether the contact results in increased Tamm-Horsfall production, an increased Tamm-Horsfall production being indicative that the molecule modulates (for example, increase) production of Tamm-Horsfall protein.

The invention further provides a method for screening for agents that modulate (for example, increase) sialylation of Tamm-Horsfall protein. The method comprises contacting Tamm-Horsfall protein in THP-positive cells with a molecule of interest and determining whether the contact results in increased sialylation of Tamm-Horsfall protein. An increase in sialylation of Tamm-Horsfall protein is indicative of modulation (for example, increase) of sialylation of Tamm-Horsfall protein.

Also provided in this invention is a pharmaceutical composition comprising Tamm-Horsfall protein and a pharmaceutically acceptable carrier.

The invention further provides a kit comprising the pharmaceutical composition that comprises the Tamm-Horsfall protein and a pharmaceutically acceptable carrier.

### BRIEF DESCRIPTION OF THE FIGURES

FIG. 1. Zeta potentials of THP from the urine of normal control subjects and IC (IC/BPS) patients show no overlap and are significantly different (P<0.002).

FIG. 2. Representative tracing of rat urodynamic studies. A, CMG showing smooth voiding contractions of a normal bladder during slow infusion of saline (40 μl/60 sec). B, CMG showing muscle reaction during KCl infusion after epithelial injury with toxic factor. As can be seen, multiple small nonvoiding contractions, a type of “fibrillation,” occur secondary to KCl. This occurs only when the epithelium loses its normal impermeability.

FIG. 3. MALDI-TOF mass spectrometry data from analysis of the polysaccharide chains of normal subjects versus IC (IC/BPS) patients. FIG. 3A depicts analysis of polysaccharide chains from IC (IC/BPS) patients. FIG. 3B depicts analysis of polysaccharide chains from normal subjects. FIGS. 3A and 3B also show schematic representation of the “tri-antennary” and “tetra-antennary” structures to the terminal part of the chains. Solid diamonds depict sialic acid. Normal subjects have significantly increased levels of heavier polysaccharide chains compared to IC (IC/BPS) patients, reflecting the increased sialic acid content.

FIG. 4. MALDI-TOF mass spectrometry of THP glycosylation chains in control subjects.

FIG. 5. MALDI-TOF mass spectrometry of THP glycosylation chains in IC (IC/BPS) subjects.

FIG. 6. Direct comparison of MALDI-TOF MS spectra for control and patient THP N-glycans.

FIG. 7. Sialic acid content of THP from the urine of IC (IC/BPS) patients (n=30) and control subjects (n=18). Each data point corresponds to the amount of sialic acid per milligram of Tamm Horsfall protein for an IC (IC/BPS) or control female. Horizontal bars represent the sample mean. No IC (IC/BPS) patient had a normal level of sialic acid. If viewed as the results of a diagnostic test, these data would reflect 95% specificity and 100% sensitivity.

FIG. 8. Direct comparison of MALDI-TOF MS spectra for control and patient THP N-glycans. Representative results of MALDI-TOF mass spectrometric analysis of the intact glycosylation chains of THP from (a) 10 control subjects and (b) 10 patients with IC/painful bladder syndrome. The N-terminal residues are depicted diagrammatically. All 10 IC (IC/BPS) patients and all 10 control subjects exhibited the same basic patterns as displayed above. Every IC (IC/BPS) patient had a preponderance of the lower molecular weight chains and little of the heavier chains that contain the tri- and tetra-antennary terminal sialic acid residues. All 10 control subjects had a preponderance of heavy chains representing primarily tri- and tetra-antennary terminal sialic acid residues.

FIG. 9. HPLC profile of 2-AB labeled N-Glycans of Normal vs. IC patient.

### DETAILED DESCRIPTION OF THE INVENTION

### Definitions

In order that the invention herein described may be more fully understood, the following description is set forth.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b. Unless otherwise indicated, all numbers expressing quantities of ingredients, properties such as molecular weight, reaction conditions, and so forth as used herein, are to be understood as being modified in all instances by the term “about.” Accordingly, unless indicated to the contrary, the numerical parameters herein are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

As used herein, “IC” refers to “Interstitial Cystitis”, “Interstitial Cystitis/Bladder Pain Syndrome” or “IC/BPS,” a progressive disorder of the lower urinary tract that causes the symptoms of urinary frequency, urgency, and/or pelvic pain in a wide variety of patterns of presentation. An example of a recent review is Parsons, Clin Obstet Gynecol, 45(1):242-249 (2002).

As used herein, “reducing,” “inhibiting”, “decreasing,” “reducing the symptoms of,” “reducing IC” and “reducing the symptoms of interstitial cystitis” refer to lowering, lessening and relieving of any one or more of the following symptoms IC: urinary urgency and frequency, and/or pelvic pain. In one embodiment, the patient may determine if interstitial cystitis symptoms are reduced. In one embodiment, reducing interstitial cystitis may be determined by the physician's evaluation. In another embodiment, reducing interstitial cystitis may be determined from comparing a Pelvic Pain and Urgency/Frequency Patient Symptom (PUF) scale score to a previous PUF scale score. In some embodiments, reducing IC/BPS involves reducing symptoms in patients whose symptoms indicate, and are similar to, interstitial cystitis.

As used herein, the term “known therapeutic compound” refers to a compound that has been shown (e.g., through animal trials or prior experience with administration to humans) to be effective in such treatment or prevention.

As used herein, the term “therapeutic” when made in reference to a compound refers to a compound that is capable of reducing, delaying, inhibiting, decreasing or eliminating one or more undesirable pathologic effects in a subject.

As used herein, “urinary frequency” refers to the number of urination times per day.

As used herein, “urinary urgency” refers to an inability to delay urination.

As used herein, “pelvic pain” refers to pain in the pelvic region of genital and non-genital origin and of organic or psychogenic aetiology.

As used herein, “urinate,” “urination,” “urinating,” “void” and “voiding” refer to release of urine from the bladder to the outside of the body.

As used herein, “urine” refers to a liquid waste product filtered from the blood by the kidneys, stored in the bladder and expelled from the body through the urethra by the act of urinating.

As used herein, “oral” and “by oral administration” refer to the introduction of a pharmaceutical composition into a subject by way of the oral cavity (e.g., in aqueous liquid or solid form).

As used herein, “oral agent” refers to a compound that can be administered by way of the oral cavity (e.g., in aqueous liquid or solid form).

As used herein, “instill” “instilled” or “instillation” refers to one or more of the following: to drop in, to pour in drop by drop, to impart gradually, to infuse slowly, to cause to be imbibed (e.g., infuse slowly an intravesical solution).

As used herein, “intravesical,” refers to inside the bladder. As such, “intravesical instillation,” “intravesical therapy,” “instill,” and “instillation” refers to solutions that are administered directly into the bladder. In some embodiments, instillation is via catheterization. Further, “intravesical solution,” “intravesical agent,” “intravesical therapeutic,” and intravesical compound” refers to a treatment that can be administered to the bladder. In one embodiment, intravesical therapy is a combination of an oral and an intravesical agent. It is not intended that the present invention be limited to a combination of an oral and an intravesical agent. For example, in one embodiment, intravesical therapy is an intravesical agent. In another embodiment, intravesical therapy is a combination of intravesical agents.

As used herein, “extravesical” refers to outside the bladder.

As used herein, “cystoscopic examination” and “cystoscopy” refers to an examination that uses a cystoscope.

As used herein, “cystoscope” refers to an endoscopic instrument to visualize the lower urinary tract, which includes the bladder and the urethra.

As used herein, “urethra” refers to a tube draining the urine to the outside. As used herein, “bladder” refers to a hollow muscular organ that stores urine until it is excreted from the body.

As used herein, the terms “subject” and “patient” refer to any animal, such as a mammal. Mammals include but are not limited to, humans, murines, simians, felines, canines, equines, bovines, porcines, ovines, caprines, rabbits, mammalian farm animals, mammalian sport animals, and mammalian pets. In many embodiments, the hosts will be humans. In one embodiment, a patient has one or more of urinary urgency, urinary frequency, pelvic pain, recurrent urinary tract infections, dyspareunia, overactive bladder, dry, etc.).

As used herein, “urinary tract infections” refers to a condition that includes an inflamed urethra and painful urination. In some embodiments, a urinary tract infection is caused by bacteria. In some embodiments, a urinary tract infection is not caused by bacteria.

As used herein, “recurrent urinary tract infections” refers to frequent episodes of urinary tract infections.

As used herein, “dyspareunia” refers to pain during intercourse.

As used herein, “overactive bladder” refers to a sudden involuntary contraction of the muscular wall of the bladder causing urinary urgency, an immediate unstoppable need to urinate and a form of urinary incontinence.

As used herein, “urinary incontinence” refers to the unintentional loss of urine and inability to control urination or prevent its leakage.

As used herein, “urinary continence” refers to a general ability to control urination.

As used herein, “catheter” refers to a tube passed through the body for draining fluids or injecting them into body cavities. It may be made of elastic, elastic web, rubber, glass, metal, or plastic.

As used herein, “catheterization” refers to the insertion of a slender tube through the urethra or through the anterior abdominal wall into the bladder, urinary reservoir, or urinary conduit to allow urine drainage.

As used herein, “catheterized” refers to the collection of a specimen by a catheterization. The terms “sample” and “specimen” are used in their broadest sense and encompass samples or specimens obtained from any source.

As used herein, the term “biological samples” refers to samples or specimens obtained from animals (including humans), and encompasses cells, fluids, solids, tissues, and gases. Biological samples include tissues (e.g., biopsy material), urine, cells, mucous, blood, and blood products such as plasma, serum and the like. However, these examples are not to be construed as limiting the types of samples that find use with the present invention.

As used herein, the term “urine cytology” refers to an examination of a urine sample that is processed in the laboratory and examined under the microscope by a pathologist who looks for the presence of abnormal cells.

As used herein, “urinary dysfunction” and “urinary tract dysfunction” refer to abnormal urination, patterns or bladder habits, including wetting, dribbling and other urination control problems.

As used herein, “anesthesia” refers to a loss of feeling or inability to feel pain.

As used herein, “local anesthesia” refers to a method of pain prevention in a small area of the body.

As used herein, the phrases “pharmaceutically acceptable salts”, “a pharmaceutically acceptable salt thereof” and “pharmaceutically accepted complex” for the purposes of this application are equivalent and refer to derivatives prepared from pharmaceutically acceptable non-toxic acids or bases including inorganic acids and bases and organic acids and bases.

As used herein, “lower urinary epithelial dysfunction” refers to disorders with positive potassium sensitivity tests (e.g., IC, prostatitis and the like). As used herein, “urinary dysfunction” refers to abnormal urination, patterns or bladder habits, including wetting, dribbling and other urination control problems.

As used herein, “sialylation” is the modification of glycoproteins with sialic acid. Sialic acid is also known as N-acetylneuraminic acid (N-acetylneuraminate). Sialic acid is often found as a terminal residue of oligosaccharide chains of glycoproteins. Sialic acid imparts negative charge to glycoproteins. In one embodiment, Tamm-Horsfall protein is sialylated.

As used herein, “repairing” involves restoring to original or close to original condition. In one embodiment of the invention, repairing the mucin layer is restoring the mucin layer of Interstitial Cystitis patients to that or normal patients. In another embodiment of the invention, repairing the mucin layer is restoring the function of the mucin layer of Interstitial Cystitis patients to that or normal patients. In a preferred embodiment, the function of the mucin layer is restored to 50% to that of normal patients. In a more preferred embodiment, the function of the mucin layer is restored to 75% to that of normal patients. In the most preferred embodiment, the function of the mucin layer is restored to 100% to that of normal patients.

As used herein, “level” of a protein is the amount of protein present. In one embodiment of the invention, the level of Tamm-Horsfall protein in a subject is the amount of Tamm-Horsfall protein present in the urine sample taken from the subject. In another embodiment of the invention, the level of sialylated Tamm-Horsfall protein in a subject is the amount of sialylated Tamm-Horsfall protein present in the urine sample taken from the subject.

As used herein, “normal” or “control” subjects are subjects who do not have clinical presentation of symptoms of Interstitial Cystitis.

In the experimental disclosure which follows, the following abbreviations apply: M (molar); mM (millimolar); μM (micromolar); nM (nanomolar); mol (moles); mmol (millimoles); μmol (micromoles); nmol (nanomoles); g (grams); mg (milligrams); μg (micrograms); pg (picograms); L (liters); mL (milliliters); ml (milliliters); μL (microliters); cm (centimeters); mm (millimeters); μm (micrometers); nm (nanometers); ° C. (degrees Centigrade).

### METHODS OF THE INVENTION

The present invention provides a method for inhibiting a condition associated with any one or more of, reduced amounts of Tamm-Horsfall protein, reduced sialylation of THP or reduced amount of carbohydrate content of Tamm-Horsfall protein, in a subject. The method comprises administering an effective amount of a Tamm-Horsfall protein to the subject. In one embodiment, THP is sialylated.

The present invention provides a method for inhibiting Interstitial Cystitis and its symptoms in a subject. The method comprises administering to the subject an effective amount of Tamm-Horsfall protein. In one embodiment, the THP protein is sialylated.

The invention further provides a method for increasing the levels of THP in a subject from existing levels of THP. The method comprises administering to the subject an effective amount of Tamm-Horsfall protein. In an embodiment of the invention, the amount of THP may be increased from about 0.1 up to 200 mg/L urine, about 0.5 up to 200 mg/L urine, about 1-5 up to 200 mg/L urine, about 5-10 up to 200 mg/L urine, about 10-15 up to 200 mg/L urine, about 15-20 up to 200 mg/L urine, about 25-30 up to 200 mg/L urine, about 30-50 up to 200 mg/L urine, about 50-75 up to 200 mg/L urine, about 75-100 up to 200 mg/L urine, about 100-125 up to 200 mg/L urine, about 125-150 up to 200 mg/L urine, about 150-175 up to 200 mg/L urine, about 0.1 up to 150 mg/L urine, about 0.5 up to 150 mg/L urine, about 1-5 up to 150 mg/L urine, about 5-10 up to 150 mg/L urine, about 10-15 up to 150 mg/L urine, about 15-20 up to 150 mg/L urine, about 20-25 up to 150 mg/L urine, about 25-30 up to 150 mg/L urine, about 30-50 up to 150 mg/L urine, about 50-75 up to 150 mg/L urine, about 75-100 up to 150 mg/L urine, about 100-120 up to 150 mg/L urine, about 120-130 up to 150 mg/L urine, about 130-140 up to 150 mg/L urine, about 0.1 up to 100 mg/L urine, about 0.5 up to 100 mg/L urine, about 1-5 up to 100 mg/L urine, about 5-10 up to 100 mg/L urine, about 10-15 up to 100 mg/L urine, about 15-20 up to 100 mg/L urine, about 20-25 up to 100 mg/L urine, about 25-30 up to 100 mg/L urine, about 30-50 up to 100 mg/L urine, about 50-60 up to 100 mg/L urine, about 60-70 up to 100 mg/L urine, about 70-80 up to 100 mg/L urine, about 80-90 up to 100 mg/L urine, 0.1 up to 50 mg/L urine, about 1-5 up to 50 mg/L urine, about 5-10 up to 50 mg/L urine, about 10-15 up to 50 mg/L urine, 15-20 up to 50 mg/L urine, about 20-25 up to 50 mg/L urine, about 25-30 up to 50 mg/L urine, about 30-40 up to 50 mg/L urine, about 40-45 up to 50 mg/L urine, about 45-50 up to 50 mg/L urine, about 0.1 up to 30 mg/L urine, about 1-5 up to 30 mg/L urine, about 0.5-5 up to 30 mg/L urine, about 5-10 up to 30 mg/L urine, about 10-15 up to 30 mg/L urine, about 15-20 up to 30 mg/L urine, about 20-25 up to 30 mg/L urine, or about 25-30 up to 30 mg/L urine. In one embodiment, THP is sialylated.

In another embodiment of the invention, increasing the levels of Tamm-Horsfall protein in a subject repairs the mucin layer of the bladder. In a further embodiment of the invention, increasing the amounts of Tamm-Horsfall protein in a subject treats diseases that are associated with decreased levels of Tamm-Horsfall protein.

As discussed in the Examples, in some patients with Interstitial Cystitis, sialylation of Tamm-Horsfall is reduced. In one embodiment of the invention, the method of inhibiting IC or reducing symptoms of IC comprises administering sialylated Tamm-Horsfall protein to a subject. Sialylation of Tamm-Horsfall protein increases the negative charge of Tamm-Horsfall protein, this making it more anionic. This results in entrapment of cations in the urine, thus inhibiting Interstitial Cystitis or reducing the symptoms of Interstitial Cystitis.

The amount of sialylation of Tamm-Horsfall protein may vary from person to person such that the Tamm-Horsfall protein may be sialylated at wild-type levels or at greater than wild-type levels. In one embodiment, wild-type levels of sialylation of THP is the amount of sialylation of THP in a sample from control or normal subjects (i.e., subjects not suffering from IC). If the Tamm-Horsfall protein is sialylated at greater than wild-type levels, a reduced dosage of sialylated Tamm-Horsfall protein may be administered to treat Interstitial Cystitis and its symptoms. As discussed infra, a person skilled in the art would determine the effective dosage of sialylated Tamm-Horsfall protein that may be administered. In a further embodiment of the invention, sialylation of Tamm-Horsfall protein may be increased from about 20-50 up to 3000 pM/μg THP, about 50-100 up to 3000 pM/μg THP, about 50-200 up to 3000 pM/μg THP, about 50-300 up to 3000 pM/μg THP, about 50-400 up to 3000 pM/μg THP, about 50-500 up to 3000 pM/μg THP, about 50-600 up to 3000 pM/μg THP, about 50-700 up to 3000 pM/μg THP, about 50-800 up to 3000 pM/μg THP, about 50-900 up to 3000 pM/μg THP, about 50-1000 up to 3000 pM/μg THP, about 50-1200 up to 3000 pM/μg THP, about 50-1400 up to 3000 pM/μg THP, about 50-1600 up to 3000 pM/μg THP, about 50-1800 up to 3000 pM/μg THP, about 50-2000 up to 3000 pM/μg THP, about 50-2200 up to 3000 pM/μg THP, about 50-2400 up to 3000 pM/μg THP, about 50-2600 up to 3000 pM/μg THP, about 50-2800 up to 3000 pM/μg THP, 20-50 up to 2500 pM/μg THP, about 50-100 up to 2500 pM/μg THP, about 50-200 up to 2500 pM/μg THP, about 50-300 up to 2500 pM/μg THP, about 50-400 up to 2500 pM/μg THP, about 50-500 up to 2500 pM/μg THP, about 50-600 up to 2500 pM/μg THP, about 50-700 up to 2500 pM/μg THP, about 50-800 up to 2500 pM/μg THP, about 50-900 up to 2500 pM/μg THP, about 50-1000 up to 2500 pM/μg THP, about 50-1200 up to 2500 pM/μg THP, about 50-1400 up to 2500 pM/μg THP, about 50-1600 up to 2500 pM/μg THP, about 50-1800 up to 2500 pM/μg THP, about 50-2000 up to 2500 pM/μg THP, about 50-2200 up to 2500 pM/μg THP, about 50-2400 up to 2500 pM/μg THP, 20-50 up to 2000 pM/μg THP, about 50-100 up to 2000 pM/μg THP, about 50-200 up to 2000 pM/μg THP, about 50-300 up to 2000 pM/μg THP, about 50-400 up to 2000 pM/μg THP, about 50-500 up to 2000 pM/μg THP, about 50-600 up to 2000 pM/μg THP, about 50-700 up to 2000 pM/μg THP, about 50-800 up to 2000 pM/μg THP, 50-900 up to 2000 pM/μg THP, 50-1000 up to 2000 pM/μg THP, 50-1200 up to 2000 pM/μg THP, 50-1400 up to 2000 pM/μg THP, 50-1600 up to 2000 pM/μg THP, 50-1800 up to 2000 pM/μg THP, 20-50 up to 1500 pM/μg THP, about 50-100 up to 1500 pM/μg THP, about 50-200 up to 1500 pM/μg THP, about 50-300 up to 1500 pM/μg THP, about 50-400 up to 1500 pM/μg THP, about 50-500 up to 1500 pM/μg THP, about 50-600 up to 1500 pM/μg THP, about 50-700 up to 1500 pM/μg THP, about 50-800 up to 1500 pM/μg THP, about 50-900 up to 1500 pM/μg THP, about 50-1000 up to 1500 pM/μg THP, about 50-1100 up to 1500 pM/μg THP, about 50-1200 up to 1500 pM/μg THP, about 50-1300 up to 1500 pM/μg THP, about 50-1400 up to 1500 pM/μg THP, 20-50 up to 1000 pM/μg THP, about 50-100 up to 1000 pM/μg THP, about 50-200 up to 1000 pM/μg THP, about 50-300 up to 1000 pM/μg THP, about 50-400 up to 1000 pM/μg THP, about 50-500 up to 1000 pM/μg THP, about 50-600 up to 1000 pM/μg THP, about 50-700 up to 1000 pM/μg THP, about 50-800 up to 1000 pM/μg THP, or about 50-900 up to 1000 pM/μg THP.

In another embodiment, the invention provides increasing the amount of Tamm-Horsfall protein in a subject so as to inhibit Interstitial Cystitis and its symptoms. For example, even if the Tamm-Horsfall protein is, partially sialylated (for example, any amount of sialic acid in a sample from a subject that is below the sialic acid content from a sample in a control subject), an increase in the total amount of the Tamm-Horsfall protein may inhibit or reduce the symptoms of Interstitial Cystitis in a subject. As discussed infra, a person skilled in the art would determine the effective dosage of the Tamm-Horsfall protein that may be administered.

In a further embodiment of the invention, an effective amount of Tamm-Horsfall protein or sialylated Tamm-Horsfall protein administered to a subject in order to inhibit or reduce symptoms of Interstitial Cystitis is about 0.1 to 200 mg/day, 0.1 to 150 mg/day, 0.1 to 100 mg/day, about 0.5 to 5 mg/day, about 5 to 50 mg/day, about 5 to 10 mg/day, about 10 to 15 mg/day, about 15 to 20 mg/day, about 20 to 25 mg/day, about 25 to 30 mg/day, about 30 to 35 mg/day, about 35 to 40 mg/day, about 40 to 45 mg/day, about 45 to 50 mg/day, about 50 to 55 mg/day, about 55 to 60 mg/day, about 60 to 65 mg/day, about 65 to 70 mg/day, about 70 to 75 mg/day, about 75 to 80 mg/day, about 80 to 85 mg/day, about 85 to 90 mg/day, about 90 to 95 mg/day, about 95 to 100 mg/day, about 2 to 10 mg/day, about 0.1 to 4 mg/day, about 0.1 to 0.5 mg/day, about 0.5 to 1.0 mg/day, about 1.0 to 1.5 mg/day, about 1.5 to 2.0 mg/day, about 2.0 to 2.5 mg/day, about 2.5 to 3.0 mg/day, about 3.0 to 3.5 mg/day, about 3.5 to 4.0 mg/day, about 4.0 to 4.5 mg/day, about 4.5 to 5.0 mg/day, about 5.0 to 5.5 mg/day, about 5.5 to 6.0 mg/day, about 6.0 to 6.5 mg/day, about 6.5 to 7.0 mg/day, about 7.0 to 7.5 mg/day, about 7.5 to 8.0 mg/day, about 8.0 to 8.5 mg/day, about 8.5 to 9.0 mg/day, about 9.0 to 9.5 mg/day, about 9.5 to 10.0 mg/day, about 0.1 to 2 mg/day, about 2 to 4 mg/day, about 4 to 6 mg/day, about 6 to 8 mg/day, about 8 to 10 mg/day, about 10 to 12 mg/day, about 12 to 14 mg/day, about 14 to 16 mg/day, about 16 to 18 mg/day, about 18 to 20 mg/day, about 0.5 mg/day, about 2 mg/day, about 10 mg/day, about 0.5 mg/day, about 0.5 to 10 mg/day, or about 0.1 to 20 mg/day. It would be clear to one skilled in the art that dosage range will vary depending on the intensity and duration of the Interstitial Cystitis symptoms. Further, it would be clear to one skilled in the art that dosage range will vary depending on the age, sex, height and/or weight of the subject and the stage at which Interstitial Cystitis is diagnosed.

In an embodiment of the invention, THP is administered directly in to the urinary tract in a subject. For example, the THP may be administered directly into the urinary tract using a catheter³⁶⁻³⁸ (Cecil Textbook of Medicine (1992) J. B. Wyngaarden et al., eds., 19^{th} ed., W.B. Saunders Co.; and Textbook of Surgery (1991) D. Sabiston, ed., 14^{th} ed., W.B. Saunders Co.). In another embodiment, the Tamm-Horsfall protein may be administered directly into the urinary tract using a time-release system. Examples of time-release systems include but are not limited to, a balloon-like device regulated to deliver a drug for 30 days, or a catheter system that is connected to a time release mechanism such as a pump, or a time release capsule inserted into the bladder. The THP administered directly in to the urinary tract of the subject may be sialylated.

The invention provides a method for diagnosing a condition associated with decreased amounts of Tamm-Horsfall protein and/or decreased amounts of sialyation of Tamm-Horsfall protein, in a subject comprising quantitatively determining in the urine from the subject, the levels of Tamm-Horsfall protein, and/or the levels of sialic acid in Tamm-Horsfall protein, and comparing the amount of Tamm-Horsfall protein and/or sialic acid, so determined to the amount in a sample from a normal subject, the decrease in the amount of Tamm-Horsfall protein and/or sialic acid being indicative of a condition associated with decreased amounts of THP and/or decreased amounts of sialyation of THP.

The invention also provides a method for diagnosing Interstitial Cystitis in a subject comprising quantitatively determining in a sample (for example, urine) from the subject, the levels of Tamm-Horsfall protein. The level of Tamm-Horsfall protein from the subject is compared to level of Tamm-Horsfall protein in a sample (for example, urine) from a control subject. A decrease in the amount of Tamm-Horsfall protein in the subject compared to that in a control subject may be indicative of Interstitial Cystitis. The level of Tamm-Horsfall protein may be determined using standard techniques including but not limited to SDS-PAGE analysis, isoelectric focusing (IEF), Western Blot Analysis, High-Performance Liquid Chromatography (HPLC), MALDI mass spectrometry and/or high pH Anion Exchange Chromatography (AEC). In control subjects, the level of THP in the sample can be, for example, in the range of about 15 μg THP/mg creatinine to about 40 μg THP/mg creatinine.

In one embodiment of the invention, diagnosing Interstitial Cystitis in a subject comprises quantitatively determining in a sample (for example, urine) from the subject, the levels of THP and the amount of sialylation of THP. The level of THP and the amount of sialylation of THP in the sample from the subject is compared to the level of THP and the amount of sialylation of THP in a sample (for example, urine) from a control subject. Both, a decrease in the amount of THP and a decrease in the amount sialylation of THP in the subject compared to that in a control subject may be indicative of Interstitial Cystitis. The amount of sialylation of THP in a sample can be determined by measuring the sialic acid content in the sample. In control subjects, the level of THP in the sample can be, for example, in the range of about 15 μg THP/mg creatinine to about 40 μg THP/mg creatinine. The sialic acid content in control subjects can be, for example, in the range of about 55 nmol/mg THP to about 85 nmol/mg THP.

In another embodiment of the invention, diagnosing Interstitial Cystitis in a subject comprises quantitatively determining in a sample (for example, urine) from the subject, the levels of THP and the amount of carbohydrates (for example, monosaccharides) in THP. The level of THP and the amount of carbohydrates (for example, monosaccharides) in THP in the sample from the subject is compared to the level of THP and the amount of carbohydrates (for example, monosaccharides) in THP in a sample (for example, urine) from a control subject. Both, a decrease in the amount of THP and a decrease in the amount of carbohydrates (for example, monosaccharides) in THP in the subject compared to that in a control subject may be indicative of Interstitial Cystitis. In one embodiment, a decrease in the amount of carbohydrates (for example, monosaccharides) in THP in a urine sample from the IC subject is a decrease in the amounts of heavy weight gycosylations chains. In another embodiment, a decrease in the amount of carbohydrates (for example, monosaccharides) in THP in a urine sample from the IC subject is a decrease in the amounts of monosaccharides in THP. In control subjects, the level of THP in the urine sample can be, for example, in the range of about 15 μg THP/mg creatinine to about 40 μg THP/mg creatinine. The carbohydrate (for example, monosaccharides) content in THP in a control subject can be, for example, in the range of about 105 nM monosaccharides/200 μg THP to about 140 nM monosaccharides/200 μg THP. In a further embodiment, a difference in the ratio of light weight glycosylation chains in THP to heavy weight glycosylation chains in THP between IC subjects and control subjects may also be used to diagnose IC. THP in samples from IC patients have reduced amounts of heavy weight glycosylation chains and therefore a higher ratio of light weight glycosylation chains to heavy weigh glycosylation chains compared to control subjects. The ratio of light weigh glycosylation chains in THP to heavy weight glycosylation chains in THP in control subjects can be, for example, less than 2.2.

The invention also provides a method for diagnosing Interstitial Cystitis in a subject by quantitatively determining in a sample (for example, urine) from the subject, the amount of sialylation of Tamm-Horsfall protein and comparing the amount of sialylation of Tamm-Horsfall protein so determined to the amount in a sample (for example, urine) from a control subject. An abnormal sialylation of Tamm-Horsfall protein in a subject compared to that in control subjects may be indicative of Interstitial Cystitis. In one embodiment, an abnormal sialylation of Tamm-Horsfall protein is a decrease in the amount of sialylation of Tamm-Horsfall protein. In another embodiment, abnormal sialylation of Tamm-Horsfall protein is the sialylation of Tamm-Horsfall protein at an alternate amino acid positions. Sialylation of Tamm-Horsfall protein can be determined by measuring the sialic acid content in the sample by using standard techniques such as Matrix-assisted laser desorption/ionization-time of flight (MALDI-TOF) mass spectrometry (MS), or measuring the zeta potential of THP, and/or by biochemically measuring sialic acid content.

In one embodiment of the invention, diagnosing Interstitial Cystitis in a subject comprises quantitatively determining in a sample (for example, urine) from the subject, the amount of sialylation of THP and the amount of carbohydrates (for example, monosaccharides) in THP. The amount of sialylation of THP and the amount of carbohydrates (for example, monosaccharides) in THP in the sample from the subject is compared to the amount of sialylation of THP and the amount of carbohydrates (for example, monosaccharides) in THP in a sample (for example, urine) from a control subject. Both, a decrease in the amount of sialylation of THP and a decrease in the amount of carbohydrates (for example, monosaccharides) in THP in the subject compared to that in a control subject may be indicative of Interstitial Cystitis. In one embodiment, a decrease in the amount of carbohydrates (for example, monosaccharides) in THP in a urine sample from the IC subject is a decrease in the amounts of heavy weight gycosylations chains. In another embodiment, a decrease in the amount of carbohydrates (for example, monosaccharides) in THP in a urine sample from the IC subject is a decrease in the amounts of monosaccharides in THP. The sialic acid content in control subjects can be, for example, in the range of about 55 nmol/mg THP to about 85 nmol/mg THP. The carbohydrate (for example, monosaccharides) content in THP in a control subject can be, for example, in the range of about 105 nM monosaccharides/200 μg THP to about 140 nM monosaccharides/200 μg THP. In a further embodiment, a difference in the ratio of light weight glycosylation chains in THP to heavy weight glycosylation chains in THP between IC subjects and control subjects may also be used to diagnose IC. THP in samples from IC patients have reduced amounts of heavy weight glycosylation chains and therefore a higher ratio of light weight glycosylation chains to heavy weigh glycosylation chains compared to control subjects. The ratio of light weigh glycosylation chains in THP to heavy weight glycosylation chains in THP in control subjects can be, for example, less than 2.2.

The invention further provides a method for diagnosing Interstitial Cystitis in a subject by quantitatively determining in a sample (for example, urine) from the subject, the total amount of carbohydrates (for example, monosaccharides) in Tamm-Horsfall protein and comparing the total amount of carbohydrates (for example, monosaccharides) in Tamm-Horsfall protein so determined to the amount in a sample (for example, urine) from a control subject. An abnormal amount of total carbohydrates (for example, monosaccharides) in Tamm-Horsfall protein in a subject may be indicative of Interstitial Cystitis. In one embodiment, an abnormal amount of total carbohydrate in THP is a decreased amount of total monosaccharides in THP. In another embodiment, a decrease in the amount of carbohydrates in THP from a sample is a decrease in the amounts of heavy weight gycosylations chains. IC (IC/BPS) patients have a deficit of heavy weight glycosylation chains compared to control subjects. Therefore, the ratio of light weight glycosylation chains to heavy weight glycosylation chains is higher in IC patients compared to control subjects. The amount of carbohydrates (for example, monosaccharides) in Tamm-Horsfall protein can be determined using standard techniques including but not limited to MALDI-TOF mass spectrometry and/or 2AB derivative analysis using High Performance Liquid Chromatography (HPLC). The carbohydrate (for example, monosaccharides) content in THP in a control subject can be, for example, in the range of about 105 nM monosaccharides/200 μg THP to about 140 nM monosaccharides/200 μg THP. The ratio of light weigh glycosylation chains in THP to heavy weight glycosylation chains in THP in control subjects can be, for example, less than 2.2.

In a further embodiment, the invention provides diagnosing Interstitial Cystitis in a subject comprising quantitatively determining in a sample (for example, urine) from the subject, the level of THP, the amount of carbohydrates (for example, monosaccharides) in THP and the amount of sialylation of THP (three-feature testing). The level of THP, the amount of carbohydrates (for example, monosaccharides) in THP and the amount of sialylation of THP in the sample from the subject is compared to the level of THP, the amount of carbohydrates (for example, monosaccharides) in THP and the amount of sialylation of THP in a sample (for example, urine) from a control subject. All three features, namely, a decrease in the level of THP, a decrease in the amount of carbohydrates (for example, monosaccharides) in THP and a decrease in the amount of sialylation of THP, in the sample from the subject compared to a sample from a control, may be indicative of IC.

Alternatively, in one embodiment of the three-feature testing, only two of the three features need be present to be indicative of IC. For example, compared to a control sample, a sample from a subject may show a decrease in the amount of carbohydrates (for example, monosaccharides) in THP, a decrease in the amount of sialylation of THP and normal levels of THP and still be indicative of IC.

One embodiment of the invention provides that in order to diagnose IC, the three features (or any two of the three features), namely, the amount of carbohydrates (for example, monosaccharides) in THP, the amount of sialylation of THP and the levels of THP, in a sample from a subject can be measured concurrently. In another embodiment, the three features (or any two of the three features), can be measured sequentially.

In one embodiment, a decrease in the amount of total carbohydrate (for example, monosaccharides) in THP is a decrease in the amount of total monosaccharides in THP. In another embodiment, a decrease in the amount of carbohydrates (for example, monosaccharides) in THP is a decrease in the amounts of heavy weight gycosylations chains. IC (IC/BPS) patients have a deficit of heavy weight glycosylation chains compared to control subjects. Therefore, the ratio of light weight glycosylation chains to heavy weight glycosylation chains is higher in IC patients compared to control subjects. In control subjects, the level of THP in the urine sample can be in the range of about 15 μg THP/mg creatinine to about 40 μg THP/mg creatinine. The carbohydrate (for example, monosaccharides) content in THP in a control subject can be in the range of about 105 nM monosaccharides/200 μg THP to about 140 nM monosaccharides/200 μg THP. The sialic acid content in a control subject can be in the range of about 55 nmol/mg THP to about 85 nmol/mg THP. The ratio of light weigh glycosylation chains to heavy weight glycosylation chains in normal subjects is approximately less than 2.2.

Also provided by the invention is a method for monitoring the course of Interstitial Cystitis in a subject. The method comprises quantitatively determining in a first sample of urine from the subject the levels of Tamm-Horsfall protein and comparing the amount so determined with the amount present in a second sample from the subject. The first and second samples are taken at different points in time and the difference in the levels of Tamm-Horsfall protein determined is indicative of the course of Interstitial Cystitis. In one embodiment of the invention, the course of Interstitial Cystitis may be graded and the grading of the diseases is based on the amount of Tamm-Horsfall protein present in a subject's urine sample, wherein the urine samples are taken at different points in time.

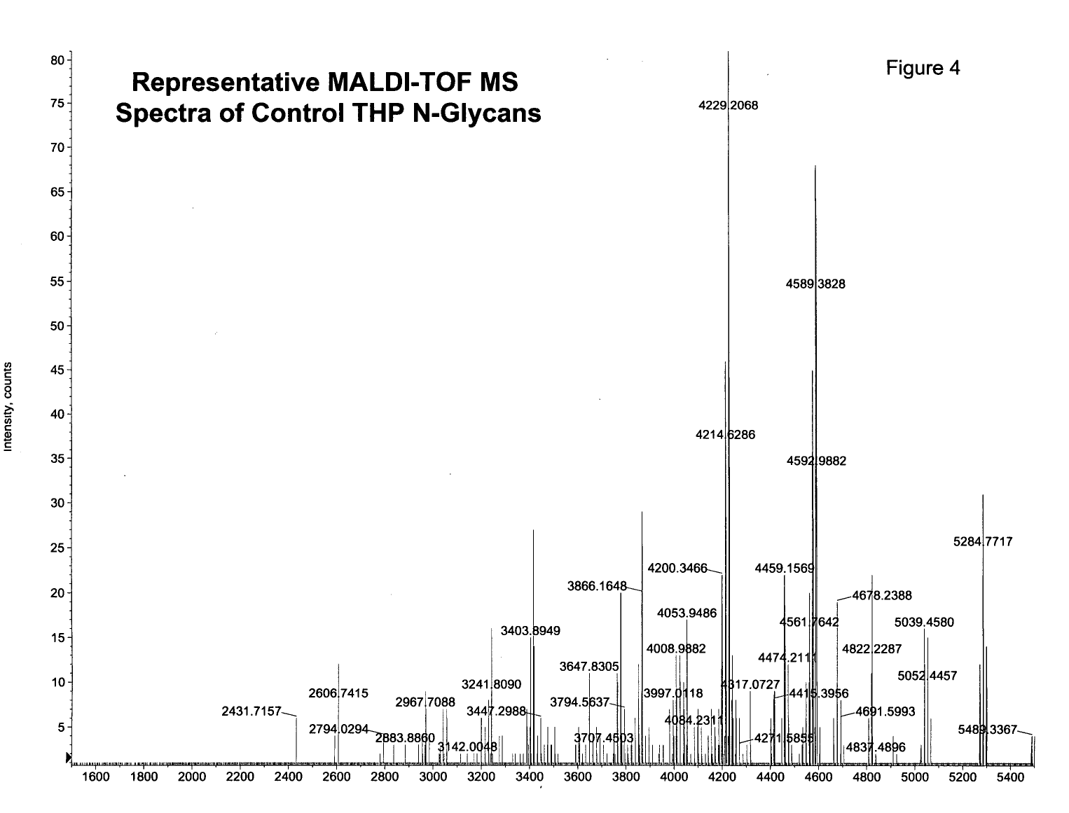

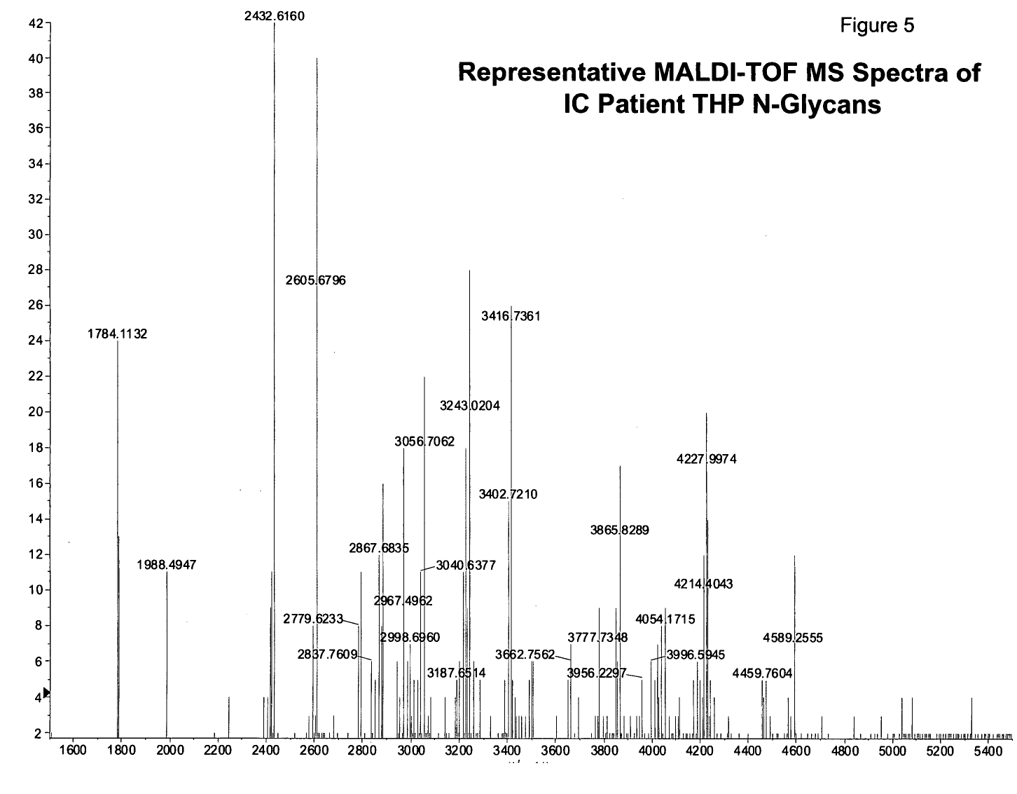

In one embodiment, the invention provides a method for monitoring the course of Interstitial Cystitis in a subject which comprises quantitatively determining in a first sample of urine from the subject the amount of sialylation of Tamm-Horsfall protein and comparing the amount so determined with the amount present in a second sample from the subject. The first and second samples are taken at different points in time. A difference in the amount of sialylation of Tamm-Horsfall protein is indicative of the course of Interstitial Cystitis. For example, decreased amount of sialylation of THP in a subject compared to normal subjects is indicative of IC (IC/BPS) (see FIGS. 4 and 5). Also, the absolute amount of THP produced by an IC (IC/BPS) patient may also be an indicator of the course of IC. Sialylation of Tamm-Horsfall protein is determined using standard techniques such as MALDI mass spectrometry and/or measuring the zeta potential of THP and/or by biochemically measuring sialic acid content.

In another embodiment, the invention provides a method for monitoring the course of Interstitial Cystitis in a subject which comprises quantitatively determining in a first sample of urine from the subject the total amount of Tamm-Horsfall protein and comparing the amount so determined with the amount present in a second sample from the subject. The first and second samples are taken at different points in time. The difference in the total amount of Tamm-Horsfall protein between the first and second samples reflects a change in the course of the disease. For example, a decrease in the total amount of Tamm-Horsfall protein produced by a subject is indicative of IC. In one embodiment, Tamm-Horsfall protein is sialylated. The total amount of Tamm-Horsfall protein is determined using standard techniques including but not limited to SDS-PAGE analysis, isoelectric focusing (IEF), Western Blot Analysis, High-Performance Liquid Chromatography (HPLC), MALDI mass spectrometry and/or high pH Anion Exchange Chromatography (AEC).

The ratios of sialic acid content in THP in control versus IC patients, total carbohydrate (for example, monosaccharides) content in THP in control versus IC patients, content of high molecular weight chains in THP in control versus IC patients and/or the total amount of THP in control versus IC patients, can be compared to set reference standards to determine normal from abnormal THP (as found in THP). For example, each normal subject should have a sufficient sialic acid content, sufficient carbohydrate (for example, monosaccharides) content in THP, sufficient content of higher molecular weight chains in THP and a minimum content of THP in the urine compared to IC/BPS patients.

In an embodiment of the invention, the subject is selected from the group consisting of human, monkey, ape, dog, cat, cow, horse, rabbit, mouse and rat subjects.

The invention also provides a pharmaceutical composition comprising Tamm Horsfall protein and a pharmaceutically acceptable carrier, known to those skilled in the art. In one embodiment, the Tamm-Horsfall protein is sialylated. The pharmaceutical compositions preferably include suitable carriers and adjuvants which include any material which when combined with the Tamm-Horsfall protein or sialylated Tamm-Horsfall protein, retain the molecule's activity, and is non-reactive with the subject's immune system.

These carriers and adjuvants include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, phosphate buffered saline solution, water, emulsions (e.g., oil/water emulsion), salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol. Other carriers may also include sterile solutions; tablets, including coated tablets and capsules. Typically such carriers contain excipients such as starch, milk, sugar (e.g., sucrose, glucose, maltose), certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres.

In one embodiment, the invention provides a kit comprising the pharmaceutical composition of the invention. In another embodiment, the invention provides a diagnostic kit for determining if a subject has IC. The diagnostic kit may be a colorimetric assay wherein when the urine sample from a subject is tested, (1) the presence, absence or reduced amount of Tamm-Horsfall protein is detected or (2) the presence, absence or reduced amount of Tamm-Horsfall activity is detected or (3) the presence, absence or reduced sialylation of Tamm-Horsfall protein is detected.

### Method for Producing Tamm-Horsfall of the Invention

The invention encompasses methods for producing Tamm-Horsfall molecules, derivatives and/or fragments thereof. The Tamm-Horsfall protein molecules, derivative and/or fragments thereof may be naturally occurring, recombinant or chemically synthesized. These may be modified by one or more purification tags, including, but not limited to, His6, epitope (e.g., myc, V5, FLAG or soft-epitope), streptavidin, biotin, avidin, tetracysteine, calmodulin-binding protein, elastin-like peptide, fusion protein (e.g., glutathione-S-transferase, maltose binding protein, cellulose-binding domain, thioredoxin, NusA or mistin), chitin-binding domain, GFP, alkaline phosphatase, cutinase, O⁶-alkylguanine alkyltransferase (AGT), or halo tag.

This method involves growing the host-vector system transfected with a plasmid encoding Tamm-Horsfall, derivatives or fragments thereof, so as to produce the Tamm-Horsfall molecules, derivatives or fragments thereof, in the host and then recovering the Tamm-Horsfall molecules, derivatives or fragments thereof. The techniques for assembling and expressing DNA encoding the amino acid sequences corresponding to Tamm-Horsfall protein, derivatives and fragments thereof, e.g., synthesis of oligonucleotides, PCR, transforming cells, constructing vectors, expression systems, and the like are well-established in the art, and most practitioners are familiar with the standard resource materials for specific conditions and procedures. The nucleotide sequences encoding the amino acid sequences corresponding to the Tamm-Horsfall protein, derivatives or fragments thereof, may be expressed in a variety of systems known in the art. The cDNA may be excised by suitable restriction enzymes and ligated into suitable prokaryotic or eukaryotic expression vectors for such expression.

Specifically, construction of suitable vectors containing the desired gene coding and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and religated in the form desired.

Site-specific DNA cleavage is performed by treating with the suitable restriction enzyme (or enzymes) under conditions which are generally understood in the art, and the particulars of which are specified by the manufacturer of these commercially available restriction enzymes (See, e.g. New England Biolabs Product Catalog). In general, about 1 μg of plasmid or DNA sequences is cleaved by one unit of enzyme in about 20 μl of buffer solution. Typically, an excess of restriction enzyme is used to insure complete digestion of the DNA substrate.

Incubation times of about one hour to two hours at about 37° C. are workable, although variations can be tolerated. After each incubation, protein is removed by extraction with phenol/chloroform, and may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in Methods in Enzymology, 65:499-560 (1980).

Restriction cleaved fragments may be blunt ended by treating with the large fragment of E. coli DNA polymerase I (Klenow) in the presence of the four deoxynucleotide triphosphates (dNTPs) using incubation times of about 15 to 25 min at 20° C. to 25° C. in 50 mM Tris (pH 7.6) 50 mM NaCl, 6 mM MgC₂, 6 mM DTT and 5-10 μM dNTPs. The Klenow fragment fills in at 5′ sticky ends but chews back protruding 3′ single strands, even though the four dNTPs are present. If desired, selective repair can be performed by supplying only one of the dNTPs, or with selected dNTPs, within the limitations dictated by the nature of the sticky ends. After treatment with Klenow, the mixture is extracted with phenol/chloroform and ethanol precipitated. Treatment under appropriate conditions with S1 nuclease or Bal-31 results in hydrolysis of any single-stranded portion.

Ligations are performed in 10-50 μl volumes under the following standard conditions and temperatures using T4 DNA ligase. Ligation protocols are standard (D. Goeddel (ed.) Gene Expression Technology Methods in Enzymology (1991)).

In vector construction employing “vector fragments”, the vector fragment is commonly treated with bacterial alkaline phosphatase (BAP) or calf intestinal alkaline phosphatase (CIP) in order to remove the 5′ phosphate and prevent religation of the vector. Alternatively, religation can be prevented in vectors which have been double digested by additional restriction enzyme digestion of the unwanted fragments.

The recombinant protein may be expressed in a prokaryotic, yeast, insect, plant or mammalian system. Examples of well known prokaryotic (bacterial) expression systems are E. coli (e.g. BL21, BL21 (DE3), XL1, XL1 Blue, DH5α or DH10B cell strains) and B. subtilis. Yeast cells include, but are not limited to, P. pastoris, K. lactis, S. cerevisiae, S. pombe, Y. lipolyt und K. marxianus. Suitable mammalian cell lines may be, among others, CHO, HEK 293 BHK, NS0, NS1, SP2/0. Insect cell lines may include, for example, Drosophila, Aedes aegypti mosquitoe, Sf21, Sf9, and T.ni cell lines. The isolated protein may comprise, depending of the expression system, different posttranslational modifications of amino acids, such as acetate groups, phosphate groups, various lipids and carbohydrates, changed chemical nature of an amino acid (e.g., citrullination) or structural changes, like disulfide bridges.

Suitable vectors include viral vector systems e.g. ADV, RV, and AAV (R. J. Kaufman “Vectors used for expression in mammalian cells” in Gene Expression Technology, edited by D. V. Goeddel (1991).

Many methods for inserting functional DNA transgenes into cells are known in the art. For example, non-vector methods include nonviral physical transfection of DNA into cells; for example, microinjection (DePamphilis et al., BioTechnique, 6:662-680 (1988)); liposomal mediated transfection (Felgner et al., Proc. Natl. Acad. Sci. USA, 84:7413-7417 (1987), Felgner and Holm, Focus, 11:21-25 (1989) and Felgner et al., Proc. West. Pharmacol. Soc., 32: 115-121 (1989)) and other methods known in the art.

### Screening Assays of the Invention

The invention provides a method for screening for agents that modulate (for example, increase) production of Tamm-Horsfall protein. The method comprises contacting Tamm-Horsfall genes in Tamm-Horsfall positive cells with a molecule of interest and subsequently determining whether the contact results in increased Tamm-Horsfall production. An increase in Tamm-Horsfall production is indicative that the molecule modulates (for example, increase) production of Tamm-Horsfall genes.

Further provided in the invention is a method for screening for agents that modulate (for example, increase) production of Tamm-Horsfall protein. The method comprises contacting Tamm-Horsfall protein in Tamm-Horsfall positive cells with a molecule of interest and then determining whether the contact results in increased Tamm-Horsfall production. An increase in Tamm-Horsfall production is indicative that the molecule modulates (for example, increase) production of the Tamm-Horsfall protein.

Also provided in this invention is a method for screening for agents that modulate (for example, increase) sialylation of Tamm-Horsfall protein. The method comprises contacting Tamm-Horsfall protein in Tamm-Horsfall positive cells with a molecule of interest and subsequently determining whether the contact results in increased sialylation of Tamm-Horsfall protein. An increase sialylation of Tamm-Horsfall protein is indicative of modulation (for example, increase) of sialylation of Tamm-Horsfall protein. In one embodiment of the invention, an increase in the sialylation of the Tamm-Horsfall protein is measured by measuring the zeta-potential of the sialylated Tamm-Horsfall protein. Examples of other methods that may be used to detect sialylation of the Tamm-Horsfall protein include but are not limited to IEF, HPLC and/or high pH anion exchange chromatography.

In one embodiment of the invention, the agents that modulate (for example, increase) sialylation of Tamm-Horsfall protein result in hyper-sialylation of the Tamm-Horsfall protein. As used herein, the term “hyper-sialylation” is sialylation of Tamm-Horsfall protein that is more than that of wild type Tamm-Horsfall protein. For example, if an active Tamm-Horsfall protein terminates in four sialic acid molecules at each sialylation site, a hyper-sialylated Tamm-Horsfall protein may terminate in more than four sialic acid residues at each sialylation site of the Tamm-Horsfall protein. Alternately, a hyper-sialylated Tamm-Horsfall protein may terminate in more than four sialic acid residues at one or more of the sialylation sites of the Tamm-Horsfall protein.

In another embodiment, the screening assay comprises mixing the recombinant-Tamm-Horsfall gene or the Tamm-Horsfall protein with a binding molecule or cellular extract. After mixing under conditions that allow association (direct or indirect) of the Tamm-Horsfall gene or the Tamm-Horsfall protein with the binding molecule or a component of the cellular extract, the mixture is analyzed to determine if the binding molecule/component increased the amount of Tamm-Horsfall protein. In one embodiment, the increase in the Tamm-Horsfall protein may be due to increased synthesis of Tamm-Horsfall protein from the recombinant Tamm-Horsfall gene. In another embodiment, the increase in the amount of Tamm-Horsfall protein may be due to increased stability or reduced degradation of Tamm-Horsfall protein. The effect of Tamm-Horsfall binding molecules may be assessed by assaying for the amount of Tamm-Horsfall protein produced, using high-throughput screening methods. Accordingly, molecules that increase the levels of Tamm-Horsfall protein can be identified.

Alternatively, targets that increase the levels of Tamm-Horsfall protein can be identified using a yeast two-hybrid system (Fields, S. and Song, O. (1989), Nature 340:245-246) or using a binding-capture assay (Harlow, supra). In the yeast two-hybrid system, an expression unit encoding a fusion protein made up of one subunit of a two subunit transcription factor and the Tamm-Horsfall protein is introduced and expressed in a yeast cell. The cell is further modified to contain (1) an expression unit encoding a detectable marker whose expression requires the two subunit transcription factor for expression and (2) an expression unit that encodes a fusion protein made up of the second subunit of the transcription factor and a cloned segment of DNA. If the cloned segment of DNA encodes a protein that binds to the Tamm-Horsfall protein, the expression results in the interaction of the Tamm-Horsfall protein and the encoded protein. This brings the two subunits of the transcription factor into binding proximity, allowing reconstitution of the transcription factor. This results in the expression of the detectable marker. The yeast two-hybrid system is particularly useful in screening a library of cDNA encoding segments for cellular binding partners of Tamm-Horsfall protein. Assaying for Tamm-Horsfall production may be used to assess the effect of the targets on the levels of Tamm-Horsfall protein.

Tamm-Horsfall proteins which may be used in the above assays include, but are not limited to, an isolated Tamm-Horsfall protein, a fragment of a Tamm-Horsfall protein, a cell that has been altered to express a Tamm-Horsfall protein, or a fraction of a cell that has been altered to express a Tamm-Horsfall protein. Further, the Tamm-Horsfall protein can be the entire Tamm-Horsfall protein or a defined fragment of the Tamm-Horsfall protein. It will be apparent to one of ordinary skill in the art that so long as the Tamm-Horsfall protein can be assayed for agent binding, e.g., by a shift in molecular weight or activity, the present assay can be used.

The method used to identify whether binding molecule and/or cellular component binds to a Tamm-Horsfall protein will be based primarily on the nature of the Tamm-Horsfall protein used. For example, a gel retardation assay can be used to determine whether an agent binds to Tamm-Horsfall or a fragment thereof. Alternatively, immunodetection and biochip technologies can be adopted for use with the Tamm-Horsfall protein. A skilled artisan can readily employ numerous art-known techniques for determining whether a particular agent increases the amount of Tamm-Horsfall protein produced.

Binding molecules and cellular components can be further tested for the ability to modulate the Tamm-Horsfall protein using a cell-free assay system or a cellular assay system. As the activities of the Tamm-Horsfall protein become more defined (for example, activities in addition to modulating Interstitial Cystitis), functional assays based on the identified activity can be employed.

As used herein, a compound/molecule is said to agonize Tamm-Horsfall activity when the compound/molecule increases Tamm-Horsfall activity by binding more cations in the urine of an IC (IC/BPS) patient or when a compound/molecule increases the amount of Tamm-Horsfall protein present in a subject or when a compound/molecule increases the sialylation of Tamm-Horsfall protein in a subject. The preferred agonist will selectively agonize Tamm-Horsfall, not affecting any other cellular proteins. Further, the preferred agonist will increase Tamm-Horsfall activity and/or levels of Tamm-Horsfall protein and/or sialylation of Tamm-Horsfall protein by more than 50%, more preferably by more than 90%, most preferably more than doubling Tamm-Horsfall activity and/or amount of Tamm-Horsfall protein and/or sialylation of Tamm-Horsfall protein.

Molecules that are assayed in the above method can be randomly selected or rationally selected or designed. As used herein, a binding molecule is said to be randomly selected when the binding molecule is chosen randomly without considering the specific sequences of the Tamm-Horsfall protein. An example of randomly selected binding molecule is the use of a chemical library or a peptide combinatorial library, or a growth broth of an organism or plant extract.

As used herein, a binding molecule is said to be rationally selected or designed when the binding molecule is chosen on a nonrandom basis that takes into account the sequence of the target site and/or its conformation in connection with the binding molecule's action. Binding molecule can be rationally selected or rationally designed by utilizing the peptide sequences that make up the Tamm-Horsfall protein. For example, a rationally selected peptide agent can be a peptide whose amino acid sequence is identical to a fragment of a Tamm-Horsfall protein.

Peptide agents can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

The binding molecule can be, for example, peptides, small molecules, and vitamin derivatives, as well as carbohydrates. A skilled artisan can readily recognize that there is no limit as to the structural nature of the agents used in the present screening method.

In one embodiment of the invention, classes of molecules that increase the levels of Tamm-Horsfall protein of the present invention are hormones. For example, in patients with Interstitial Cystitis, symptoms of Interstitial Cystitis are reduced during pregnancy. In a further embodiment of the invention, estrogen is administered to a subject to stimulate the production of Tamm-Horsfall protein. In yet another embodiment of the invention, progesterone is administered to the subject to stimulate production of Tamm-Horsfall protein.

The cellular extracts embodied in the methods of the present invention can be, as examples, aqueous extracts of cells or tissues, organic extracts of cells or tissues or partially purified cellular fractions. A skilled artisan can readily recognize that there is no limit as to the source of the cellular extract used in the screening method of the present invention.

The method for determining whether a molecule or a compound causes an increase in the amount of Tamm-Horsfall protein comprises separately contacting each of a plurality of samples to be tested according to any of the methods of the invention. In one embodiment, the plurality of samples may comprise, more than about 10⁴ or more than about 5×10⁴ samples. In another particular embodiment, the method comprises essentially simultaneously screening the molecules according to any one of the described methods of the invention.

The screening assays of the present invention for identifying candidate agents can, e.g., detect incorporation of a label, where the label can directly or indirectly provide a detectable signal. Various labels may be used, include radioisotopes, fluorescers, chemiluminescers, and the like.

A variety of other reagents may be included in the screening assay. These include reagents like salts, detergents, neutral proteins, e.g., albumin, etc., that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4° C. and 40° C. Incubation periods are selected for optimum activity, but may also be optimized to facilitate rapid high-throughput screening.

### Pharmaceutical Compositions

The present invention provides compositions that are useful in treating IC (IC/BPS) and/or its symptoms, including pharmaceutical compositions, comprising the THP polypeptides, polynucleotides or other molecules of the invention. The compositions may include a buffer, which is selected according to the desired use of the THP polypeptide, polynucleotides or other molecules of the invention, and may also include other substances appropriate to the intended use. Those skilled in the art can readily select an appropriate buffer, a wide variety of which are known in the art, suitable for an intended use. The compositions may also include a biodegradable scaffold, matrix or encapsulating material such as liposomes, microspheres, nanospheres and other polymeric substances. In some instances, the composition can comprise a pharmaceutically acceptable carrier or excipient, a variety of which are known in the art and need not be discussed in detail herein. Pharmaceutically acceptable excipients have been amply described in a variety of publications, including, for example, Gennaro, A. R.

(2003) Remington: The Science and Practice of Pharmacy with Facts and Comparisons: Drugfacts Plus. 20^{th} ed., Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed., Amer. Pharmaceutical Assoc. In some embodiments, the composition comprises a matrix that allows for slow release of the composition.

The pharmaceutically acceptable excipients, such as vehicles, adjuvants, carriers, and diluents, are readily available to the public. Moreover, pharmaceutically acceptable auxiliary substances, such as pH adjusting and buffering agents, tonicity adjusting agents, stabilizers, wetting agents and the like, are readily available to the public.

The THP polynucleotides and polypeptides may be obtained from naturally occurring sources or synthetically or recombinantly produced. Where obtained from naturally occurring sources, the source chosen will generally depend on the species from which the protein is to be derived. The subject proteins may also be derived by synthesis, such as by synthesizing small fragments of a polypeptide and later linking the small fragments together. The subject protein can be more efficiently produced by recombinant techniques, such as by expressing a recombinant gene encoding the protein of interest in a suitable host, whether prokaryotic or eukaryotic, and culturing such host under conditions suitable to produce the protein. If a prokaryotic host is selected for production of the protein, such as E. coli, the protein will typically be produced in and purified from the inclusion bodies. If an eukaryotic host is selected for production of the protein, such as CHO cells, the protein may be secreted into the culture medium when its native or a heterologous secretory leader sequence is linked to the polypeptide to be made. Any convenient protein purification procedures may be employed. Suitable protein purification methodologies are described in Guide to Protein Purification, Deuthser ed. (Academic Press, 1990). For example, a lysate may be prepared from the original source and purified using HPLC, exclusion chromatography, gel electrophoresis, affinity chromatography, and the like.

The molecules of the invention can be formulated into preparations for delivery by dissolving, suspending or emulsifying them in an aqueous solvent, such as phosphate buffered saline (PBS), or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The molecules of the invention can be provided in unit dosage forms, i.e., physically discrete units suitable as unitary dosages for human and animal subjects, each unit containing a predetermined quantity of molecules of the present invention calculated in an amount sufficient to produce the desired effect in association with a pharmaceutically acceptable diluent, carrier, or vehicle. The specifications for the novel unit dosage forms of the present invention depend on the particular molecule/compound employed and the effect to be achieved, and the pharmacodynamics associated with each compound in the host.

An effective amount of the molecules of the invention (for example, a subject polypeptide of the invention) is administered to the subject at a dosage sufficient to produce a desired result.

Typically, the compositions of the instant invention will contain from less than about 1% to about 95% of the active ingredient (molecules of the invention), in some embodiments, about 10% to about 50%. Administration can be generally by catheterization and often to a localized area. The frequency of administration will be determined by the care giver based on patient responsiveness. Other effective dosages can be readily determined by one of ordinary skill in the art through trials establishing dose response curves.

In order to calculate the amount of molecules of the invention to be administered, those skilled in the art could use readily available information with respect to the amount of agent necessary to have the desired effect. The amount of a molecule necessary to increase a level of active subject polypeptide can be calculated from in vitro or in vivo experimentation. The amount of agent will, of course, vary depending upon the particular agent used and the condition of the subject being treated, such as the subject's age, the extent of the subject's disease, the subject's weight and the likelihood of any adverse effect, etc.

Regarding pharmaceutical dosage forms, the therapeutic agents may be administered in the form of their pharmaceutically acceptable salts, or they may also be used alone or in appropriate association, as well as in combination, with other pharmaceutically active compounds or treatment procedures. The following methods and excipients are merely exemplary and are in no way limiting.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Gennaro, A. R. (2003) Remington: The Science and Practice of Pharmacy with Facts and Comparisons: Drugfacts Plus. 20^{th} ed., Lippincott, Williams, & Wilkins. The composition or formulation to be administered will, in any event, contain a quantity of the therapeutic agent adequate to achieve the desired state in the subject being treated.

The compositions of the invention will be formulated and dosed in a fashion consistent with good medical practice, taking into account the clinical condition of the individual subject, the site of delivery of the polypeptide composition, the method of administration, the scheduling of administration, and other factors known to practitioners. The effective amount of polypeptide for purposes herein is thus determined by such considerations.

The following examples are presented to illustrate the present invention and to assist one of ordinary skill in making and using the same. The examples are not intended in any way to otherwise limit the scope of the invention.

### EXAMPLES

Urine contains low molecular weight cationic factors called “toxic factors” (TF) and these factors are capable of injurying normal bladder mucus and initiating the symptoms of IC. The toxic factors are neutralized by THP. In IC (IC/BPS) patients, the ratio of the toxic factors to THP is increased. The capacity of THP to neutralize the toxic factors is dependent on the sialic acid content of the molecule. The THP of IC (IC/BPS) patients has reduced capacity to neutralize these factors as shown in cell culture cytotoxicity assays and a rodent urodynamic model. The neutralizing capacity of THP from IC (IC/BPS) patients is reduced due to the lower sialic acid content. The quantity of THP needed in IC (IC/BPS) patients to neutralize the toxic factors is increased when compared to the quantity of THP required in normal subjects.

The structure of the glycosylation chains of the THP molecule in IC (IC/BPS) patients is different compared to normal subjects. The glycosylation chains in normal subjects terminate in N-sialic acid which branch to form tertiary and quaternary structures (antennae) for many of the chains while the IC (IC/BPS) patients have a reduced number of these tertiary and quaternary antennae.

### Example 1

Normal urine contains low molecular weight (LMW) cations toxic to cultured bladder cells and that Tamm-Horsfall protein (THP), a ubiquitous urinary glycoprotein, protects against this cytotoxic effect. THP may sequester and subsequently neutralize these toxic urinary cations. In this study we tested whether the presence of THP can limit the injury caused by these urinary cations using an in vivo rat bladder injury model.

### Materials and Methods

### Human and Animal Subjects

Collection of Urine from Healthy Human Volunteers. Informed consent was obtained and 24-hour pooled urine samples were obtained from 3 healthy female volunteers (mean age 30 years). After collection, urines were stored at −20 EC until use for isolation of the toxic factor (TF).

Animal subjects. Animal studies employed adult male Sprague-Dawley (SD) rats under a protocol approved by the Veterans Affairs Medical Center Institutional Animal Care and Use Committee.

Preparation of the Toxic Factor or Dialyzed Product from Human Subjects

Potential human control subjects were screened for IC (IC/BPS) symptoms using the Pelvic Pain and Urgency/Frequency Patient Symptom (PUF) Scale.⁹ A PUF Score of 0 was required for study entry.

The LMW (>100<3500) toxic factor (TF; dialysis product) was prepared by dialyzing 500 ml of a 24-hr urine sample from control subjects in a dialysis bag (MWCO 100) (Spectrum Laboratories, Inc., Rancho Dominguez, Calif.) against distilled water until chloride ion was no longer detectable in the dialysate (outside) with 0.05 M AgCl solution. At that time, the dialyzed urine was placed in another dialysis membrane (MWCO 3500) and the overnight-dialyzed product (<3500 MW) lyophilized and investigated after rehydration (10 mg/ml) for its ability to induce bladder contractions.

### Cytotoxicity Assay

The toxic factor (>100<3500 MWCO) was tested for cytotoxicity using the CellTiter96 Aqueous One Solution Proliferation Assay™ (Promega Corp, Madison, Wis.). Rat urothelial target cells and human HBT4 urothelial cells were plated in 96-well tissue culture plates (Nunclon™) in triplicate (50000 cells/well). Cells had been maintained in Ham's-M199 tissue culture media supplemented with 10% fetal calf serum (FCS)+antibiotics (Pen-Strep).

For cytotoxicity assay, cells were harvested and resuspended in DME media (without phenol red indicator) with 1% FCS (assay media) containing 100 μg of solubilized TF in a volume of 100 μl/well. Negative control wells were similarly prepared but did not contain any TF. For the wells containing TF plus THP, TF 2 mg/ml was mixed with THP 2 mg/ml and incubated for 1 hour at room temperature and then sedimented in a centrifuge and the supernatant (100 μl) added to triplicate test wells.

After overnight incubation at 37° C., the wells were washed twice with DME alone (200 μl/well) and fresh DME added to each well (90 μl). Then a novel tetrazolium compound (MTS-Owen's Reagent) combined with an electron coupling reagent (PES) was added (10 μl/well) per kit protocol and absorbance measured in a plate reader after 30 minutes incubation at 490 nm and at 630 nm after blanking the plate to DME media containing the tetrazolium compound. The background at 630 nm was subtracted from the 490 nm reading to determine percentage cytotoxicity relative to the media controls.

Cytotoxicity levels were compared between groups as follows: TF alone versus control, TF plus THP versus TF alone, and TF plus THP versus control.

### Evaluation of Bladder Overactivity in Rodents

After a few days of acclimatization, two groups (n=9 each) of adult male Sprague-Dawley rats (325-350 g) underwent urodynamics evaluation of bladder hyperactivity before and after intravesical infusion of a test substance (TF or TF plus THP) followed by intravesical KCl.⁸ The procedure was as follows.

The rats were anesthetized with a subcutaneous injection of urethane (1.2 g/Kg) and a 1 cm incision was made along the centerline of the lower ventral abdomen. Once the bladder was exteriorized, a 22 gauge (0.7 mm) catheter (Intracath, Becton-Dickinson, OH) was inserted into the bladder dome and sutured in place using a purse string suture with 4.0 tapered prolene suture. The bladder was returned to the abdomen, with the line escaping through the incision. The muscle wall was sutured together using 4.0 tapered prolene suture and the skin was sutured using 4.0 nylon suture. The catheter was then connected to a pressure transducer (UFI, Morro Bay, Calif.) and in turn connected to an infusion pump (Harvard Apparatus, Mass.). During the continuous filling bladder cystometry, the pressure was recorded with the transducer using the program LabView (National Instruments, TX).

To induce hyperactivity, the bladder was first infused with warm (37° C.) 0.9% saline or 400 mM KCl (29.8 mg/mL, Abbott Laboratories, IL) at 40 μL/min (2.4 mL/hr) and at least 20 minutes of stable voiding cycles were recorded during infusion. The pressure threshold (PT, pressure at which voiding initializes) and peak pressure (PP, pressure maximum or amplitude of bladder contractions) were recorded. Frequency of contractions and inter-contractile interval were recorded, along with the percentage of non-voiding contractions (% NVC, contractions where the PP is greater than 2 cm H₂O and less than the PT, thus not resulting in a void).

After these baseline measurements, rats were infused with the test solution. To test the hypothesis that TF could injure the bladder mucosa and facilitate KCl-induced bladder hyperactivity, one group of animals received an infusion of TF (15 mg/mL). To test the hypothesis that THP could attenuate the bladder hyperactivity induced by TF and KCl, a second group of animals received an infusion of a mixture of THP 10 mg/ml and TF (15 mg/ml) mixed in a ratio of 1:1.

In both groups, infusion of the test solution was followed by infusion of 400 mM KCl (29.8 mg/mL, Abbott Laboratories, IL) infusion for 30 minutes⁸ and all urodynamics measurements were repeated.

### Statistical Analysis

Results were analyzed by Student's t test, employing the Bonferroni correction where more than two variables were involved.

### Results

### Results of the In Vitro Cytotoxicity Assays

The TF had a significant cytotoxic effect in cultured rat (p<0.01) and human (p<0.01) HBT4 bladder epithelial cells relative to the negative control. In the wells containing TF plus THP, there were no detectable levels of cytotoxicity (Table 1).

### Results of In Vivo Rat Studies

Infusion of intact rat bladder with NaCl or KCl resulted in normal and comparable numbers of voids and NVC (Table 2). After intravesical TF, KCl infusion resulted in a significant increase in NVC relative to pre-TF values (p<0.0001). After infusion of TF premixed with THP, KCl irrigation was associated with significantly lower numbers of NVC than those seen after infusion of TF alone (p<0.0001). There was no significant difference in the numbers of NVC recorded at baseline and the number recorded after infusion of TF plus THP (Table 2).

### DISCUSSION

It was previously proposed that IC (IC/BPS) results from a disruption in the critical balance of protective mechanisms and potentially pathogenic factors in the lower urinary tract.³ It is recognized that the relative impermeability of the bladder mucosa is the primary protective mechanism of the bladder wall. There is substantial evidence that bladder epithelial permeability is regulated by the surface mucus.¹⁰⁻¹² Data from a number of centers indicate that IC (IC/BPS) is associated with an epithelial dysfunction that results in abnormal permeability.^{1, 2, 13-17}

Data from earlier studies indicate that the primary urinary toxin that appears to be initiating symptoms and tissue injury is the cation potassium.^{1, 2, 18-21} Potassium is present in relatively high levels (60-130 mEq/L) in the urine; when the epithelium is abnormally permeable, potassium is allowed to diffuse down the gradient from high urine levels to lower tissue levels, where it can depolarize nerves and muscle and, on a chronic basis, injure tissue.^{1, 2, 18} When the epithelial permeability barrier is intact, potassium does not provoke symptoms.¹

Recent investigations have focused on the possible role of urine factors in the pathogenesis of IC. In earlier studies, it was found that normal human urine contains LMW cations similar to protamine sulfate, a cationic compound known to be extremely toxic to the mucus.⁴ These LMW cations, partially isolated in the toxic factor (TF), are likely to be amines that are the end product of protein metabolism. It has been previously reported that the TF causes epithelial cell injury in vitro.⁴ The cationic TF have the potential for electrostatically binding to the anionic transitional cell surface mucus, disrupting the permeability barrier and allowing the cascade of potassium diffusion, sensory nerve stimulation, and tissue injury to occur.^{1, 2, 18} The presence of a urinary toxic factor has also been proposed by Keay, who isolated an antiproliferative factor that may have a role in epithelial injury.²²

The difference between a healthy state and the disease state may be a qualitative or quantitative deficiency of a protective factor that normally prevents the cations from injuring mucus by electrostatically attracting them before they have a chance to damage the bladder surface. In previous studies, it has been proposed that THP may function as a urinary protector that prevents the natural byproducts of metabolism from injuring mucus.^{4, 7} We have shown that THP is capable of neutralizing these toxic factors in a cell culture assay.⁷

We conducted the current study to verify the presence of the urinary toxic factors and to test the ability of THP to neutralize their toxic effect both in vitro and in vivo. To accomplish this, we used two models. First, we isolated a LMW fraction from urine and used an in vitro cytotoxicity assay to determine its cytotoxicity to cultured urothelial cells and to test for potential neutralization of the toxic effects by prior incubation with THP. We found that the urinary TF causes significant cytotoxicity. Exposure of the TF to THP, which is highly anionic, abrogated the toxic effect of this LMW fraction (Table 1).

Second, we used a rodent urodynamics model⁸ in which the bladders of healthy rats are infused with KCl before and after urothelial injury with protamine sulfate (PS) and a marked increase in muscle reactivity is observed in response to KCl after the PS treatment. We substituted TF for PS as the epithelial injury agent in this model. A rat with a healthy urothelium should not react to KCl, and our baseline KCl data confirmed this, showing no significant increase in bladder hyperactivity in animals treated with KCl versus NaCl. When the urothelium was injured with TF, however, we found a marked increase in NVC due to altered urothelial permeability to KCl. The NVC represent muscle spasticity or fibrillation, an abnormal reaction to KCl. We found THP blocked the NVC induced by the TF (Table 2). While these are animal and cell culture models, the data do support the concept that these functions of both human TF and THP are operating in the fashion described above in the human bladder.

The findings from the current study lend support to our hypothesis that THP may exert a protective effect in the bladder. THP may operate in the urine by electrostatically binding potentially injurious cationic urine factors that might otherwise injure the urothelium. If THP's function is inadequate, then the resultant increase in urothelial permeability may allow urinary potassium, a passive player in the pathogenetic process, to penetrate the tissue and activate bladder nerves and muscle.

Both our earlier data and the results of the current study support the hypothesis that the initiation of IC (IC/BPS) may rest in the imbalance of cations and anions in urine, with THP being the primary anion responsible for protecting the epithelium.^{4, 7} We have isolated a cationic toxic urinary fraction and shown that it is as injurious as protamine to the epithelial cells of the bladder, resulting in increased permeability and increased sensitivity to potassium.⁴ Further, our data indicate that THP can neutralize both protamine⁷ and the anionic urinary toxic factor.⁴

These findings suggest that THP may operate to bind potentially injurious cations in the urine in healthy individuals. If this is the case, then the ability of THP to perform this function may contribute to the prevention of IC (IC/BPS) in the normal state. Conversely, the pathogenesis of IC (IC/BPS) may involve a reduction in the protective capacity of THP.

The initiating event for IC (IC/BPS) may be a normal protein metabolite which, if left unchecked or if present in sufficient concentrations, injures the urothelium by electrostatically binding to the mucus, altering the permeability of the epithelium, and resulting in K diffusion and tissue response of nerve depolarization, injury, and inflammation. THP, a highly anionic urinary protein, electrostatically neutralizes the injurious effects of this toxic urine factor. THP appears to play a protective role in the normal urinary tract, and a reduction in its protective effect could initiate the cascade of urothelial injury, increased urothelial permeability, and potassium diffusion that we have hypothesized for the pathogenetic process of IC.

TABLE 1
Cytotoxicity levels in rat and human cultured urothelial cells
after treatment with urinary toxic factor or toxic factor plus THP*

| | Human cells (HBT4) | | | | | |
|---|---|---|---|---|---|---|
| | | Cyto- | | Rat urothelial cells | | |
| | | toxicity | Statistical | | Cytotoxicity | Statistical |
| Group | N | (%) | significance | N | (%) | significance |
| Control | 9 | 0 | — | 9 | 0 | — |
| (negative) | | | | | | |
| TF | 9 | 27.1 | p < 0.01† | 9 | 31.1 | p < 0.01† |
| TF + THP | 9 | 0 | p < 0.01‡ | 9 | 0 | p < 0.01‡ |

Key:
THP, Tamm-Horsfall protein;
TF = toxic factor
*We used this cytotoxicity assay to verify the presence of the toxic factor (TF). Clearly, the urine contained TF and the TF was neutralized by THP. This toxic urine fraction was used in the in vivo rat model.
†Compared to control.
‡Compared to TF alone. There was no significant difference between cytotoxicity levels in the control group and those in the group treated with TF plus THP.

TABLE 2
Effect of potassium on rat bladder after treatment with urinary toxic
factor or toxic factor plus THP*

| Agent infused into rat | | | Statistical |
|---|---|---|---|
| bladder | N | NVC/minute | significance |
| NaCl (baseline) | 9 | 0.2400 ± 0.074 | — |
| KCl (baseline) | 4 | 0.25 ± 0.064 | — |
| Toxic factor | 9 | 1.681 ± 0.11 | p < 0.0001† |
| Toxic factor plus | 9 | 0.2778 ± 0.08462 | p < 0.0001‡ |
| THP | | | |

Key:
THP, Tamm-Horsfall protein;
NVC, nonvoiding contractions;
TF = toxic factor
*As can be seen, similar to the cytotoxicity results, the TF injured the rat urothelium and allowed K to diffuse into the interstitium, where it provoked NVC (“fibrillation” activity); the TF was neutralized by THP.
†Compared to NaCl baseline value.
‡Compared to TF alone. No significant difference when compared to NaCl baseline value.

### Example 2

Tamm-Horsfall protein (THP) from normal urine has been shown to protect against the cytotoxic effects of toxic urinary cations (TF) in vivo and in vitro. This study investigated the effect of desialylation on the ability of THP to protect the urothelium in vivo and in vitro from the effects of a urine-derived toxic factor (TF). Desialylation would reduce the electronegativity of the proteins, impairing its effectiveness for attracting the cationic TF.

### Materials and Methods

### Human and Animal Subjects

Healthy female volunteers (median age 30 years) provided 24-hour pooled urine samples. These subjects were screened for IC (IC/BPS) symptoms using the Pelvic Pain and Urgency/Frequency Patient Symptom (PUF) Scale.⁹ A PUF Score of 0 was required for study entry to ensure that THP and TF were obtained from the urine of healthy individuals who had no evidence of bladder disease or voiding symptoms. Urines were stored at −20° C. until they were used for isolating TF and/or THP.

Animal studies employed adult male Sprague-Dawley (SD) rats weighing 325-350 g.

### Preparation of THP and THP-d

THP was prepared from urines by the method of Tamm and Horsfall.⁶ Briefly, THP was recovered by centrifugation after precipitation in the cold overnight with 0.6M NaCl. The gel-like precipitate was resuspended in 50 ml cold 0.6M NaCl, then reprecipitated by centrifugation. This was repeated three times to increase the purity of the final product which was dissolved in a minimal amount of distilled water, pH 7.4. The solubilized THP was exhaustively dialyzed to remove all traces of salt and then lyophilized. Dry weight of this material was subsequently used to prepare stock solutions of THP (10 mg/ml) dissolved in PBS (in vivo studies) or culture media (cell studies). THP preparations were monitored for purity by PAGE and identification of THP made by Western blot.

Desialylated THP (THP-d) was prepared by mild acid hydrolysis of THP (10 mg/ml) in 2.5M acetic acid and heating for 3 hr at 82° C. The THP hydrolysate was neutralized by filtration-washing, three times with 15 ml PBS, on a Centricon (MWCO 30000) cartridge (Millipore). After each wash the volume was reduced by centrifugation to about 1 ml. The recovered desialylated protein (>30000 MW) in the last wash (2 ml final volume) was devoid of free sialic acid which appeared in the washes and could be quantitated by DMB derivitization and fluorescent detection to assess the extent of desialylation. The hydrolysis resulted in 87.7% loss of sialic acid (16.12 vs 1.99 μg Neu5Ac/mg protein for the THP-d), coincident with an increase in the electrophoretic migration of the THP.

Preparation of the Toxic Factor from Human Subjects

The LMW (>100<3500) TF was prepared as described previously.²³

### Cytotoxicity Assay

The TF obtained from 2 different pooled urine samples (TF1, TF2) was tested for cytotoxicity using the CellTiter96 Aqueous One Solution Proliferation Assay™ (Promega Corp, Madison, Wis.). Rat urothelial target cells and human HTB4 urothelial cells were plated in 96-well tissue culture plates (Nunclon™) in triplicate (50,000 cells/well). Cells had been maintained in Ham's-M199 tissue culture media supplemented with 10% fetal calf serum (FCS)+antibiotics (Pen-Strep).

For cytotoxicity assay, cells were harvested from seed flasks, washed free of trypsin-EDTA, and resuspended in DME media (without phenol red indicator) −1% FCS (assay media). An aliquot of the cell suspension was counted in a hemocytometer and diluted with assay media to a concentration of 0.5×10⁶ cells/ml, from which 100 μl (5×10⁴ cells) were added to each well in a 96-well microtiter plate. Test samples (100 μl) were added to these wells in quadruplicate. For these assays, THP (obtained from 4 different normal urines) was pooled together, and the desialylated THP from these same samples (THP-d) were mixed with TF and incubated at room temperature for 60 min, then centrifuged before they were added to the wells containing the target cells. Samples of each test material were tested individually for cytotoxicity and tested in checkerboard fashion with the THP, THP-d, and TF preparations. Dose response data was determined beforehand for the TF preparations. The final concentrations of all test samples were adjusted so that they were identical during assay.

After overnight incubation at 37° C., the target cells were washed twice with DME alone (200 μl/well) and fresh DME added to each well (90 μl). Then a tetrazolium compound (MTS-Owen's Reagent) combined with an electron coupling reagent (PES) was added (10 μl/well) per kit protocol and absorbance measured in a plate reader after 30 min incubation at 490 nm and at 630 nm after blanking the plate to DME media containing the tetrazolium compound. The background at 630 nm was subtracted from the 490 nm reading to determine percentage cytotoxicity relative to the media controls.

### Evaluation of Bladder Overactivity in Rodents

After a few days of acclimatization, adult male SD rats underwent urodynamics evaluation of bladder hyperactivity before and after intravesical infusion of a test substance⁸ (TF or TF plus unmodified THP or TF plus THP-d) followed by intravesical KCl. The procedure for the in vivo rat study was performed as described previously.²³

After baseline measurements of pressure threshold, peak pressure, frequency of contractions, inter-contractile interval, and percentage of nonvoiding contractions (NVC) were recorded as previously described,²³ rats were infused with the test solution. One group of animals received an infusion of TF (15 mg/mL). To test the effect of desialylation on the ability of THP to attenuate the bladder hyperactivity induced by TF and KCl, second and third groups of animals received an infusion of a mixture of unmodified THP or THP-d 10 mg/ml and TF (15 mg/ml) mixed in a ratio of 1:1.

Infusion of the test solution was followed by infusion of 400 mM KCl for 30 min⁸ and urodynamics measurements were repeated.

### Statistical Analysis

Results were analyzed by Student's t test, employing the Bonferroni correction where more than two variables were involved.

### Results

### Results of the In Vitro Cytotoxicity Assays

TF had a significant cytotoxic effect on HTB4 epithelial cells relative to the media controls (P<0.01). In the wells containing TF mixed with unmodified THP, there were no detectable levels of cytotoxicity (Table 3). In the wells containing incubated with THP-d, however, there was significant cytotoxicity (13.4%), approximately the same (P=0.5) as when TF alone was incubated with the target cells (average 7.4%) (Table 3).

### Results of In Vivo Rat Studies

Infusion of intact rat bladder with NaCl or KCl resulted in normal and comparable numbers of voids and NVC (Table 4). After intravesical TF, KCl infusion resulted in a significant increase in NVC relative to pre-TF values (P<0.0001). After infusion of TF premixed with unmodified THP, KCl irrigation was associated with significantly lower numbers of NVC than those seen after infusion of TF alone (P<0.0001). There was no significant difference in the numbers of NVC recorded at baseline and the number recorded after infusion of TF plus unmodified THP (Table 4). After infusion of TF mixed with THP-d, KCl irrigation was associated with a significantly higher NVC rate than that seen after infusion of TF plus unmodified THP (P<0.0001).

### DISCUSSION

IC (IC/BPS) may be the result of a disruption in the balance of protective factors and potentially pathogenic factors in the lower urinary tract.³ There is substantial evidence that the relative impermeability of the bladder mucosa is the primary mechanism that protects the bladder wall and that the surface mucus is critical in regulating epithelial permeability.^{10, 12} Data from a number of investigators indicate that IC (IC/BPS) is associated with an epithelial dysfunction resulting in abnormal permeability.^{1, 13, 14, 16, 17}

Studies indicate that the cation potassium is the chief urinary toxin to initiate the symptoms and tissue injury of IC.^{1, 9, 18-21} Potassium is present in relatively high levels in the urine (20-120 mEq/l).¹⁸ An abnormally permeable epithelium permits potassium to diffuse into the bladder tissue, where it can depolarize nerves and muscle and produce tissue injury.^{1, 18} Potassium in the bladder does not provoke symptoms when the epithelial permeability barrier is intact.¹

In recent studies, urine factors have been investigated for their role in IC (IC/BPS) pathogenesis. These data show that normal human urine contains LMW cations, or TFs, that cause epithelial cell injury in vitro and in vivo.^{4, 23, 7} The TFs are similar to protamine sulfate, a cationic compound known to be extremely toxic to the mucus of the epithelium, causing a permeability abnormality.¹² It is likely that these TFs are amines or polypeptides that are the end product of protein metabolism. Potentially, they can bind electrostatically to the anionic transitional cell surface mucus and disrupt the permeability barrier. This disruption permits the cascade of potassium diffusion, sensory nerve stimulation, muscle depolarization, and tissue injury.^{1, 18}

The difference between a healthy state and the disease state in the lower urinary tract may be a qualitative or quantitative deficiency of a factor that normally protects the epithelium from injury by urinary constituents. Such a protective factor might function by electrostatically attracting the potentially toxic cations before they can damage the bladder surface. Epithelial injury, then, is initiated when there is an imbalance between the urinary TF and the protective factors. On the basis of earlier data, we have proposed that THP functions as a urinary protective factor to prevent the TF, which are natural byproducts of metabolism, from injuring the bladder epithelium.^{4, 23, 7} Our data indicate that these TF are neutralized by THP.^{23, 7}

In the current study, we isolated a LMW TF fraction from urine, verified its cytotoxicity to cultured urothelial cells, and tested the ability of unmodified and desialylated THP to neutralize the toxic effects in vitro and in vivo. The data indicate that the urinary TF causes significant cytotoxicity. The toxic effect of this LMW fraction was neutralized by exposure to unmodified THP but not by exposure of the TF to THP that had undergone desialylation (Table 3).

Second, the ability of unmodified versus desialylated THP to neutralize the toxic effects of the urinary TF in vivo using was investigated using a rodent urodynamics model.⁸ In our adaptation of this model, the bladders of healthy rats are infused with KCl before and after urothelial injury with urinary TF, and the resulting muscle reactivity (NVC) is quantified. The data confirmed that a rat with a healthy urothelium does not exhibit an increase in NVC in response to intravesical KCl, but a rat who has undergone urothelial injury with TF does display a marked increase in NVC in response to KCl because the urothelial permeability has been altered. Exposure to THP appeared to prevent TF from inducing urothelial injury, as would be evidenced by a rise in NVC (Table 4). When TF was exposed to desialylated THP, however, there was a significant rise in NVC, suggesting that desialylated THP lacks the protective effects of unmodified THP.

The data presented herein that THP exerts a protective effect in the bladder. Urinary THP may electrostatically bind cationic urine factors and prevent them from injuring the anionic urothelium. THP samples do not all have equal ability to perform this protective function.⁷ Consequently, such urine factors may then injure the urothelium, increase urothelial permeability, and allow urinary potassium to penetrate the tissue and stimulate bladder nerves and muscle in certain individuals. Such “abnormal” THP may result from aberrant sialylation pathways leading to decreased sialic content of the N-(or O-linked) glycans present on THP.²⁴ The results of the current study support the hypothesis that IC (IC/BPS) may result from the disruption of a balance between protective anionic factors and potentially injurious cations in the urine.^{4, 23, 7} These results demonstrate a cationic toxic urinary fraction that is similar to protamine in its ability to injure the epithelial cells of the bladder, resulting in increased permeability and increased sensitivity to potassium.^{4, 23, 7} THP neutralizes the injurious effects of both protamine⁷ and the cationic urinary toxic factor.^{4, 23} The current study indicates that the protective activity of THP is dependent on sialic acid, a highly anionic part of the THP molecule that most likely imparts the protective activity to the protein.

These findings suggest that THP is a critical urinary anion that protects the epithelium, acting to bind potentially injurious cations in the urine in healthy individuals. If THP does indeed play this protective role in the urine, its ability to do so may be a major factor in the prevention of IC (IC/BPS) in the normal state. Conversely, a reduction in the protective capacity of THP may be an important factor in the pathogenesis of IC. This concept opens new vistas in the diagnosis and therapy of IC, including determining the susceptibility to IC (IC/BPS) by detecting the presence of desialylated THP.

THP, a highly anionic urinary protein, electrostatically neutralizes the injurious effects of toxic urine factor. This activity appears to depend on the anionic terminal sialic acid moieties on the THP molecule. THP appears to play a protective role in the normal urinary tract, and a reduction in its protective effect could initiate the cascade of urothelial injury, increased urothelial permeability, and potassium diffusion for the pathogenetic process of IC.

TABLE 3
Cytoprotection of target cells against toxic factor by unmodified
versus desialylated Tamm-Horsfall protein*

| | | Average | Statistical | |
|---|---|---|---|---|
| | | cytotoxicity | significance: test | |
| Test substance | N | (%)† | vs. media alone | Result |
| Media | 8 | 0 | — | — |
| TF | 8 | 7.4 | P < 0.01 | Toxic |
| THP + TF:‡ | | | | |
| THP + TF | 32 | 0 | NS | Not Toxic |
| THP-d + TF | 32 | 13.4 | P < 0.01 | Toxic |

Key:
TF = toxic factor;
THP = Tamm-Horsfall protein;
THP-d = desialylated Tamm Horsfall protein;
n = number of wells assayed;
NS = not significant.
*Target cells were human bladder cells (HTB4)-5 × 10⁴/well
†Cytotoxicity of protamine sulfate, a known cytotoxic agent: LD50 = ~20 μg
‡Cytotoxicity of THP-d + TF (all) vs. TF (1, 2) not significant (P = 0.5)

TABLE 4
Effect of potassium on rat bladder after treatment
with urinary toxic factor, toxic factor plus unmodified Tamm-Horsfall
protein, or toxic factor plus desialylated Tamm-Horsfall protein*

| Agent infused into rat bladder | n | NVC/minute | P value |
|---|---|---|---|
| NaCl (baseline) | 9 | 0.24 ± 0.074 | — |
| KCl (baseline) | 4 | 0.25 ± 0.064 | — |
| TF | 9 | 1.68 ± 0.11 | P < 0.0001† |
| TF + unmodified THP | 14 | 0.42 ± 0.13 | P < 0.0001‡ |
| TF + THP-d | 6 | 1.55 ± 0.62 | P < 0.0001§ |

Key:
NVC = nonvoiding contractions;
TF = toxic factor;
THP = Tamm-Horsfall protein;
THP-d = desialylated Tamm Horsfall protein.
*As can be seen, similar to the cytotoxicity results, the TF allowed K to diffuse into the interstitium, where it provoked NVC (“fibrillation” activity); the TF effects were abolished by THP.
†Compared to NaCl baseline value.
‡Compared to TF alone. No significant difference when compared to NaCl baseline value.
§Compared to TF + unmodified THP.

### Example 3

This example demonstrates the measurement of the sialic acid content of THP from the urine of normal subjects versus IC (IC/BPS) patients. To investigate a possibility that would lend additional support to the concept that sialic acid content is reduced in IC (IC/BPS) versus normal subjects, the zeta potential of the THP molecule was measured, which would be reduced in IC (IC/BPS) patients if the sialic acid content is lower. Urinary THP concentrations in IC (IC/BPS) patients versus normal control subjects was also determined to rule out the possibility that IC (IC/BPS) patients produce lower quantities of THP to account for a reduction of protective activity.

### Materials and Methods

Isolation of THP from Urines

THP is isolated by the cartridge centrifugation-washing method. For this isolation method, two 15-ml aliquots of urine are centrifuged sequentially until they result in a 30× concentration. The protein fraction >30000 MW (top) is then washed by 2 centrifugations with 15 ml distilled water. The final material containing the THP (>95%) is brought up to 1 ml volume with water containing 0.01% azide preservative and stored at 4° C. until ELISA or sialic acid determination. The filtrated urine does not contain any traces of protein by detection with Coomassie® Brilliant Blue-G250 reagent (Bio-Rad Laboratories, Hercules, Calif.). Before use, representative THP samples are monitored and characterized for purity by PAGE and Western blot identification. This method allows almost 100% recovery of the protein and partial purification by washing and removes all traces of salt.

### Hydrolysis of THP and Sialic Acid Determination

An aliquot (150 μl volume) of the THP recovered from normal (n=29) and IC (IC/BPS) (n=28) urines by filtration-concentration, is subjected to mild acidic hydrolysis (2.5M acetic acid) by heating at 80° C. for 3 hr. The free sialic acid in the hydrolysate is recovered by passing through minicon filters (10000 MW cutoff) to remove any protein, then dried with mild heat under vacuum centrifugation and finally sent for sialic acid determinations by the DMB fluorescent detection method (sensitivity pM range). THP samples prior to hydrolysis were shown to have no detectable endogenous sialic acid.

The dried THP samples are dissolved in 50 μl of milliQ water and 50 μl of 7 mM DMB (1,2-diamino-4,5-methylenedioxybenzene dihydrochloride) in acetic acid added. The samples are warmed to 50° C. for 2.5 hr and without any further purification 20 μl of the derivatized sialic acids are injected for HPLC using a reversed phase C18 column (TosoHaas ODS-120T). A gradient of water:acetonitrile:50% methanol starting at 79:7:14 then changing to 75:11:14 over 40 min elution time is used which separates the various acetylated species. The fluorescence detector is set at Ex.373 nm and Em.448 nm. Quantitation is accomplished by comparing peak heights to known amounts of purified standards derivatized during the same run. The amount of sialic acid in a 150 μl hydrolysate of the THP samples is then used to calculate the amount of sialic acid pM/μg THP in the normal- and IC-derived THP samples. Differences in the sialic acid content of the IC (IC/BPS) and normal THP (mean THP/group) are compared by Student's t test.

### ELISA Assay for THP Quantitation

THP isolated from urines is assayed by an indirect enzyme-linked immunosorbent assay (ELISA) using 96-well plastic plates (Immulong, Thermo Electron, Waltham, Mass.). Test plates are coated with purified THP (Biomedical Technologies, Inc., Stoughton, Mass.) 100 ng/well by incubating overnight in 0.05 M carbonate coating buffer (pH 9.6) at 4° C. and then washing with PBS 2× and blocked in PBS+0.5% BSA-0.01% Tween-20 for 1 hr at room temperature. Wells are washed in PBS-0.01% Tween-20 (assay buffer, pH 7.4) and used immediately after washing with distilled water or stored after being dried. For quantitation of THP by the indirect ELISA, a standard curve is prepared by adding 100 μl of twofold serial dilutions of a THP standard (2.000-0.015 ng/well) to duplicate wells and immediately adding 100 μl of a 1/2000 dilution of goat anti-THP (ICN Pharmaceuticals, Inc., Costa Mesa, Calif.) to each well. A THP control, no THP (buffer alone), is prepared as above and mixed with the antibody. Samples are assayed in duplicate, 10 μl of each sample is added to 90 μl of assay buffer and 100 μl of the goat anti-THP added (1:2000) and the plate(s) incubated overnight on a shaker at room temperature. The assay plate is washed 3× with assay buffer, then 100 μl of second antibody, rabbit anti goat-peroxidase (Sigma Chemical Co., St. Louis, Mo.) in assay buffer at 1:1000 dilution added for 1 hr. The plates are washed, and OPD peroxidase substrate (Sigma Chemical Co.) added for exactly 10 min in the dark, and the reaction stopped with dilute HCl. The plates are blanked to substrate in the plate reader and read at 450 nm. Average amounts of THP in the duplicate samples are extrapolated from the standard curve and the concentrations recorded as μg THP/150 μl (hydrolysate) or mg THP/L urine. THP is also normalized to urinary creatinine. Student's t test is used to determine whether there is any significant difference in the THP concentration in IC (IC/BPS) patients' vs. normal urines.

### THP Zeta Potential (mV) Measurements

Surface charge properties (zeta potential) are measured for THP samples from patients (n=6) and controls (n=6). THP (Biomedical Technologies, Inc.) and fetuin and asialofetuin (Sigma Chemical Co.) standards (50 μg) are similarly measured. Zeta potential and size measurements are made with a Zetasizer Nano ZS instrument and a CGS-3 Goniometer system (Malvern Instruments Inc., Southborough, Mass.) with ancillary software version 4.0. All zeta potential measurements are conducted on rehydrated samples in 0.01M NaCl buffer, pH 7.0 at 25° C., after centrifugation followed by filtration (0.45 μM) to remove any aggregates. Measurements are made in triplicate and the results analyzed by Student's t test for significance.

Urines were collected from female IC (IC/BPS) patients (median age 30 years) who were screened for IC (IC/BPS) symptoms using the Pelvic Pain and Urgency/Frequency Patient Symptom (PUF) Scale (23). Control urines were obtained from healthy individuals who had no evidence of bladder disease, bladder irritative voiding symptoms or clinical history suggestive of recent urinary tract infection and who had a PUF Score of 0. In accordance. with institutional review board policy, informed consent was obtained prior to collection of all samples.

Urines were collected for two purposes: (1) for quantitative determination of urinary levels of THP in IC (IC/BPS) patients compared to normal individuals and the determination of the sialic acid content (pM sialic acid/μg THP protein) of the THP and (2) to provide larger (mg) amounts of THP for determination of zeta potentials. Fresh morning urine voids collected in the UCSD Urology Clinic usually provide ˜30 ml of urine. These can be further processed by a rapid, filtration-washing protocol that can process 1-12 urine samples daily, providing purified THP (>95%) for further characterization by ELISA and sialic acid determinations described below. To provide sufficient THP for zeta potential determinations requiring 5 mg/ml purified THP, 24-hour urine samples were obtained from some IC (IC/BPS) and control subjects.

THP was isolated from control subjects' and IC (IC/BPS) patients' urine; subjected to hydrolysis and sialic acid determination, measurement of surface charge properties (zeta potential); and quantitated by ELISA as described in the Supporting Online Material.

### Results

There were 29 normal subjects and 28 IC (IC/BPS) patients. Sialic acid content was significantly lower in IC (IC/BPS) patients' THP than in control subjects' THP (224 vs. 1001 pM/μg THP; P<0.01, t test) (Table 5).

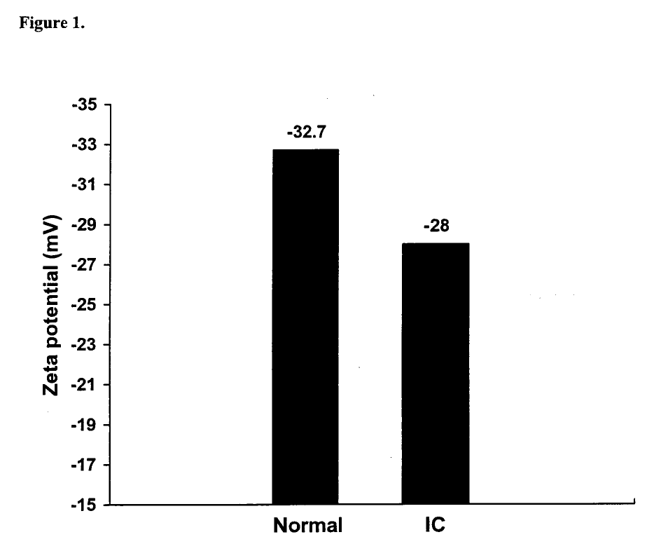

IC (IC/BPS) patients' THP had a significantly lower zeta potential than control THP (−32.7±1.4 vs. −28.0±2.4 mV; n=6 in each group; P<0.002, t test), a difference of 14.4% (FIG. 1). These results indicate IC (IC/BPS) patients' THP has significantly less surface charge (electronegativity).

THP concentrations in normal urine (28.2 mg/L) and IC (IC/BPS) urine (28.8 mg/L) were not significantly different. When THP was normalized to creatinine, there was no significant difference between the normal and IC (IC/BPS) patients in urinary THP concentration (76.4 versus 70.0 mg THP/mg creatinine) (Table 6).

### DISCUSSION

Prior experiments have demonstrated that bladder surface mucus plays a critical role in controlling the permeability of the epithelium, principally to small molecules^{10-12, 25, 26}. If the mucus is impaired, it results in a dysfunctional epithelium that allows movement of concentrated urinary solutes into the bladder interstitium^{1-3, 13-16, 18}. One solute, potassium (K+), is 10- to 40-fold more concentrated in urine than in tissue. If there is a dysfunctional epithelium, K+ moves readily down that gradient into the bladder interstitium, where it can directly depolarize nerves, muscles, and generate the symptoms of frequency, urgency, pain, and urinary incontinence, in any combination^{1, 3, 8, 17, 18, 27, 28}. There is extensive evidence that this diffusion of potassium occurs in IC, and it is a phenomenon that could account for the entire cascade of symptom generation and tissue injury seen in this disease^{1-3, 8, 17, 18, 27, 28}.

The key question is why this dysfunctional epithelium occurs. It is recognized that the glycosaminoglycans in the healthy epithelial mucus are extremely hydrophilic. This “hydrating effect” traps water at the cell surface and inhibits the movement of molecules across the epithelium^{10, 30-32}. In the gastrointestinal tract, this layer of water has been called the “unstirred layer of water”³³⁻³⁵. When the bladder mucus is exposed to highly charged cationic compounds such as protamine sulfate, the mucus is rendered dysfunctional, resulting in a “leaky epithelium”¹⁰. Because the epithelial mucus is highly anionic, naturally occurring urinary cations potentially could bind to it and alter its ability to hydrate the cell membrane. To test this concept, a low molecular weight cationic compound(s) called toxic factor (TF) was isolated from normal urine. TF and protamine sulfate (highly cationic) injure urothelial cells in vitro and alter epithelial permeability in vivo, resulting in potassium diffusion into the bladder muscle and an increase in nonvoiding bladder muscle contractions^{23, 29}.

The next logical question is whether urine contains protective molecules that are anionic and capable of electrostatically attracting TF (and protamine) and neutralizing their toxic effects on the epithelium. It appears to have just such a molecule, Tamm-Horsfall protein (THP). The interaction of THP with cationic urine molecules results in sequestering these potentially toxic ions to the oppositely charged layer surrounding THP. Serum factors that are protein bound to albumen likewise become biologically ineffective; an example is testosterone. A similar activity is proposed for THP; in this case, urinary toxic factors are bound. In both instances, the phenomenon can be mediated by surface charge, or the zeta potential. THP, manufactured by the renal tubular cells, is a large (85 kD) molecular weight protein that is highly anionic. It is a well-conserved protein present in all vertebrate species^{6, 5}, but in effect has no known obvious urinary activity. THP may thus be a scavenger of the TF, that it is abnormal in IC (IC/BPS) patients compared to healthy individuals.

THP is able to detoxify the TF in vivo and in vitro^{4, 23, 7}. IC (IC/BPS) patients' THP has a lower cytoprotective activity than normal subjects' THP against the known toxic effects of protamine sulfate⁵. The sialic acid content of THP imparts a substantial amount of the electronegativity to the molecule. THP from IC (IC/BPS) patients has lower protective activity than THP from normal subjects against another cation, PS, in vitro⁷.

The results of this example support our hypothesis that THP is abnormal in IC (IC/BPS) patients and that the abnormality is a significant reduction in THP sialic acid content. Sialic acid content was significantly lower (approximately 80%) in IC (IC/BPS) patients than in normal controls. In addition, the zeta potential, which represents the electronegativity of the molecule, was significantly lower in THP from IC (IC/BPS) patients than in THP from normal control subjects. Such a difference could result in the loss of THP's ability to sequester the TF, ultimately resulting in mucosal injury and epithelial dysfunction leading to the entire cascade that results in IC.

Because a lower total urinary THP concentration could account for the finding of lower sialic acid content and zeta potential in IC (IC/BPS) patients' THP, the total THP concentration in the urine from normal subjects and IC (IC/BPS) patients was determined. There were no differences between the normal and the IC (IC/BPS) samples regardless of whether THP concentration was determined in terms of mg protein per liter of urine or mg protein per mg creatinine (Table 6).

These findings may represent an important piece of the puzzle of IC (IC/BPS) pathogenesis. It appears that THP of IC (IC/BPS) patients is abnormal, allowing the TF in urine to cause lower urinary dysfunctional epithelium (LUDE)³ and begin the entire IC (IC/BPS) cascade of neurologic upregulation, muscle hyperactivity, tissue injury, and inflammation. These data may have important implications not only for understanding of the initial cause of IC (IC/BPS) but also for knowledge of physiologic role of THP in urine, a role that has not been well understood. With the understanding that a defect in a urinary protective molecule (THP) can lead to substantial problems in the health of the urinary bladder, efforts can be focused on new vistas in IC (IC/BPS) therapy. These might include creating THP analogs to replace the defective THP, developing methods for correcting the THP defect, or using strategies such as dietary changes to decrease the concentration of urinary TF. The finding of a specific abnormality in IC (IC/BPS) patients' THP, coupled with the evidence that normal THP operates as an important protective factor against bladder epithelial injury, represents a major step in understanding the etiology of IC (IC/BPS) and developing diagnostic and therapeutic treatments for IC.

TABLE 5
Sialic acid content in urinary THP from IC (IC/BPS) patients versus
normal controls. THP, Tamm-Horsfall protein; IC, interstitial cystitis.

| | | Sialic acid content | |
|---|---|---|---|
| Group | N | (pM/μg THP) | P value |
| Control subjects | 27 | 964 | |
| IC | 25 | 224 | <0.001* |
| (IC/BPS)patients | | | |

*Versus control.

TABLE 6
Total THP in IC (IC/BPS) versus normal urine.
Urinary THP

| mg/L urine | | | mg/mg creatinine | | |
|---|---|---|---|---|---|
| Normal | IC | | Normal | IC | |
| n = 39 | n = 115 | P Value | n = 25 | n = 76 | P Value |
| 28.2 | 28.8 | N.S. | 76.4 | 70.0 | P = 0.596 |

THP, Tamm-Horsfall protein;
IC, interstitial cystitis;
N.S., not significant.

### Example 4

This example was performed to determine whether the toxic factor is capable of changing the permeability characteristics of the intact bladder epithelium in vivo. A more reliable cytotoxicity assay was used, less subject to artifact errors caused by manipulating target cells during the wash steps and incubations used in earlier assays, to screen urine fractions for cytotoxicity and to measure the ability of pentosan polysulfate (PPS) to neutralize the cytotoxic activity of the toxic factor or protamine sulfate (PS). In addition, a new in vitro rat urodynamic model⁴ was used to investigate the effect on induced bladder contractions of exposure of the normal bladder epithelium to KCl alone versus urinary toxic factor followed by KCl. This new model allows quantitation of bladder muscle reactivity under experimental conditions. Nonvoiding contractions (NVC) of the bladder represent muscle spasticity or fibrillation that is an abnormal reaction to KCl. Protamine sulfate (PS) was used as a positive control in both models. PS will bind to the glycosaminoglycans (GAGs) of the mucus and disrupt its permeability regulatory mechanism.⁵ This provides a good model for potential disease in the bladder relative to making the epithelium dysfunctional. Urine is likely to contain natural “protamine-like” cations also capable of injuring the epithelium. Consequently, the urinary toxic factor was evaluated and compared to our positive control (PS) for its ability to injure cultured urothelial cells as well as an intact rodent urothelium in vivo. The mechanism by which PPS can potentially control the symptoms of IC (IC/BPS) was also explored. It was determined whether the highly anionic structure of PPS can electrostatically bind to charged toxic cations before they can injure the mucosa. Heparinoids may have similar reactivity in their successful use in management of IC.

### Materials and Methods

### Human and Animal Subjects

Collection of urine from healthy human volunteers. Informed consent was obtained and 24-hour pooled urine samples obtained from 3 healthy female volunteers, median age 30 years. After collection, urines were stored at −20° C. until use. The anonymity of the human subjects was preserved in accordance with institutional policies and the Health Insurance Portability and Accountability Act (HIPAA) of 1996.

Animal subjects. Animal studies employed adult male Sprague-Dawley (SD) rats under a protocol approved by the Veterans Affairs Medical Center Institutional Animal Care and Use Committee.

Preparation of the Toxic Factor or Dialyzed Product from Human Subjects

Human control subjects were screened for IC (IC/BPS) symptoms using the Pelvic Pain and Urgency/Frequency Patient Symptom (PUF) Scale.⁶ A PUF Score of 0 was required for study entry.

The LMW (>100<3500) toxic factor was prepared by dialyzing 500 ml of a 24-hr urine sample from control subjects in a dialysis bag (MWCO 100) (Spectrum Laboratories, Inc., Rancho Dominguez, Calif.) against distilled water until chloride ion was no longer detectable in the dialysate (outside) with 0.05 M AgCl solution. At that time, the dialyzed urine was placed in another dialysis membrane (MWCO 3500) and the overnight-dialyzed product (<3500 MW) lyophilized and investigated after rehydration (10 mg/ml) for its ability to induce bladder contractions.

### Cytotoxicity Assay

The toxic factor (>100<3500 MWCO) was tested for cytotoxicity using the CellTiter96 Aqueous One Solution Proliferation Assay™ (Promega Corp, Madison, Wis.),^{7, 8} adapted for use with cultured rat urothelial cells and our test substance, the toxic factor derived from urine by dialysis as described above. Rat urothelial target cells isolated from rat epithelium were plated in 96-well tissue culture plates (Nunclon™) in triplicate (50000 cells/well). Cells had been maintained in Ham's-M199 tissue culture media supplemented with 10% fetal calf serum (FCS)+antibiotics (Pen-Strep).

For cytotoxicity assay, cells were harvested and resuspended in DME (without phenol red indicator) with 1% FCS (assay media) containing 100 μg of solubilized toxic factor in a volume of 1 μl/well. Negative control wells were similarly prepared but did not contain any toxic factor. Positive control wells contained protamine sulfate (PS), 1 mg/ml. For PS+PPS or toxic factor+PPS, PS (2 mg/ml) or toxic factor 2 mg/cc was premixed with an equal volume of PPS (2 mg/ml) and incubated for 1 hour, then sedimented in a centrifuge and the supernatant (100 μl) added to triplicate test wells.

After overnight incubation at 37° C., the wells were washed twice with DME alone (200 μl/well) and fresh DME added to each well (90 μl). Then a novel tetrazolium compound (MTS-Owen's Reagent) combined with an electron coupling reagent (PES) was added (10 l/well) per kit protocol and absorbance measured in a plate reader after 30 minutes incubation at 490 nm and at 630 nm after blanking the plate to DME media containing the tetrazolium compound. The background at 630 nm was subtracted from the 490 nm reading to determine percentage cytotoxicity relative to the media controls.

Cytotoxicity levels were compared between groups as follows: positive control (PS) versus negative control; toxic factor alone versus negative control; toxic factor plus PPS versus toxic factor alone; and PS plus PPS versus positive control.

### Evaluation of Bladder Overactivity in Rodents

After a few days of acclimatization, three groups (n=9 each) of adult male Sprague-Dawley rats (325-350 g) underwent urodynamics evaluation of bladder hyperactivity before and after intravesical infusion of a test substance (PS, urinary toxic factor, or toxic factor plus PPS) followed by intravesical KCl.⁴

The procedure was as follows: The rats were anesthetized with a subcutaneous injection of urethane (1.2 g/Kg) and a 1 cm incision made along the centerline of the lower ventral abdomen. Once the bladder was exteriorized, a 22 gauge (0.7 mm) catheter (Intracath, Becton-Dickinson, OH) was inserted into the bladder dome and sutured in place, using a purse string suture with 4.0 tapered prolene suture. The bladder was returned to the abdomen, with the line escaping through the incision. The muscle wall was sutured together using 4.0 tapered prolene suture and the skin was sutured using 4.0 nylon suture. The catheter was then connected to a pressure transducer (UFI, Morro Bay, Calif.) and in turn connected to an infusion pump (Harvard Apparatus, Mass.). During the continuous filling bladder cystometry, the pressure was recorded with the transducer using the program LabView (National Instruments, TX).

To induce hyperactivity, the bladder was first infused with warm 0.9% saline (37° C.) at 40 μL/min (2.4 mL/hr) and at least 20 minutes of stable voiding cycles recorded during infusion. The pressure threshold (PT, pressure at which voiding initializes) and peak pressure (PP, pressure maximum or amplitude of bladder contractions) were recorded. Frequency of contractions and inter-contractile interval (ICI) were recorded, along with the percentage of non-voiding contractions (% NVC, contractions where the PP is greater than 2 cm H₂O and less than the TP, thus not resulting in a void).

After these baseline measurements, rats were infused with the test solution. Group 1 (positive control) animals received 1 mL of a warm solution (37° C.) of 30 mg/mL PS (Sigma-Aldrich, St. Louis, Mo.) for 30 minutes. PS has been shown to significantly injure urothelial permeability.⁵ To test the hypothesis that urinary toxic products could injure the bladder mucosa and facilitate KCl-induced hyperactivity, Group 2 animals received an infusion of toxic factor (15 mg/mL). To test the hypothesis that PPS could attenuate the bladder hyperactivity induced by toxic product and KCl, Group 3 animals received an infusion of a mixture of PPS (10 mg/ml) and toxic factor (15 mg/ml).

In all three groups, infusion of the test solution was followed by infusion of 400 mM KCl (29.8 mg/mL, Abbott Laboratories, IL) infusion for 30 minutes⁴ and all urodynamics measurements were repeated.

### Statistical Analysis

Results were analyzed by Student's t test, employing the Bonferroni correction where more than two variables were involved.

### Results

### Results of the In Vitro Cytotoxicity Assays

Both PS (the positive control) and the toxic factor had a significant (p<0.001) cytotoxic effect in cultured rat bladder epithelial cells relative to the negative control. In the wells containing toxic factor plus PPS, however, cytotoxicity levels were significantly lower than in the wells containing toxic factor alone (p<0.007). The combination of PS and PPS was associated with significantly lower rates of cytotoxicity than PS alone (p<0.02) (Table 7).

### Results of In Vivo Rat Studies

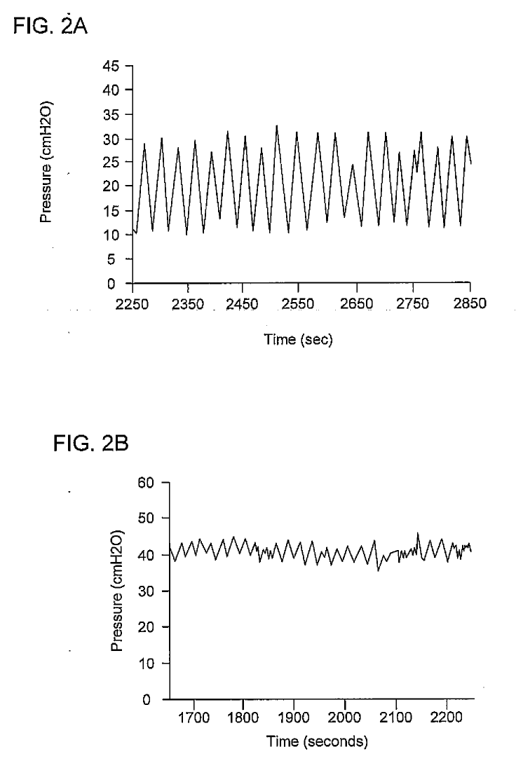

Infusion of intact rat bladder with sodium or KCl resulted in normal and comparable numbers of voids (1.240±0.1875/min) and NVC (FIG. 2A; Table 8). After intravesical PS or toxic factor (FIG. 2B), KCl infusion resulted in a significant increase in NVC relative to pre-PS/toxic factor values. After infusion of toxic factor premixed with PPS, KCl irrigation was associated with significantly lower numbers of NVC than those seen after infusion of toxic factor alone. There was no significant difference in the numbers of NVC recorded at baseline and the number recorded after infusion of toxic factor plus PPS (Table 8).

In this in vitro cytotoxicity assay, data was obtained indicating that a cytotoxic factor is present in urine and has a toxic effect on cultured urothelial cells. Pentosan polysulfate was demonstrated to neutralize the cytotoxic effect of the urinary toxic factor as well as of the positive control protamine sulfate.

TABLE 7
Cytotoxicity levels in rat cultured urothelial cells after treatment with
urinary toxic factor or toxic factor PPS

| | | Cytotoxicity | Statistical |
|---|---|---|---|
| Group | N | (%) | significance |
| Control (negative; no TF or PS) | 9 | 0 | — |
| Protamine sulfate (positive control) | 9 | 61.9 ± 27.6 | p < 0.001† |
| Toxic factor | 12 | 26.9 ± 13.9 | p < 0.001† |
| Toxic factor plus PPS | 9 | 10.9 ± 8.6 | p < 0.007‡ |
| Protamine sulfate plus PPS | 9 | 0 | p < 0.02§ |

†Compared to negative control group.
‡Compared to toxic factor alone.
§Compared to positive control group.

In this in vivo study, infusion of potassium into the bladder provoked “fibrillation” activity, as indicated by a significant increase in nonvoiding contractions, after the bladder had been treated with either the urinary toxic factor or the known epithelial injury agent protamine sulfate. Potassium did not cause fibrillation in an untreated bladder, suggesting the toxic factor injures the urothelium and allows K to diffuse into the interstitium, where it activates the muscle and nerves. As in the in vitro assay, pentosan polysulfate neutralizes the injurious effect of the urinary toxic factor.

TABLE 8
Effect of potassium on rat bladder after treatment with urinary
toxic factor or toxic factor plus PPS

| Agent infused into rat | | | Statistical |
|---|---|---|---|
| bladder | N | NVC/minute | significance |
| NaCl (baseline) | 9 | 0.2400 ± 0.074 | — |
| KCl (baseline) | 4 | 0.25 ± 0.064 | — |
| Group 1: Protamine sulfate | 9 | 1.733 ± 0.55 | p = 0.01† |
| (positive control) | | | |
| Group 2: Toxic factor | 9 | 1.681 ± 0.1131 | p = 0.0004† |
| Group 3: Toxic factor plus PPS | 9 | 0.63 ± 0.05 | p = 0.0102‡ |

†Compared to NaCl baseline value.
‡Compared to Group 2. No significant difference when compared to NaCl baseline value.

### DISCUSSION

Normal human urine was investigated to determine whether it contained compounds capable of causing epithelial cell injury, possibly by interfering with the protective function of the mucus lining of the bladder, leading to epithelial injury and loss of the permeability barrier function. This would allow access of urinary solutes to the bladder interstitium, leading to initiation of symptoms and potentially to cell injury. Urine also has been shown to contain many secondary factors that are a result of the disease process and include inflammatory mediators, neurotransmitters, growth factors or even an antiproliferative factor.^{9, 10} The occurrence of these factors in urine from patients is mostly of a secondary nature, produced by the disease and not acting as primary inciting factor. Nevertheless, some of these factors may be involved in tissue reactions and disease progression.

The results presented herein demonstrate that urine contains LMW cations similar to PS, which has been shown to be a toxic, mucus-damaging substance.⁵ These cations, present in urine, have the potential for electrostatically binding to the anionic transitional cell surface mucus, disrupting the permeability barrier and allowing the cascade of potassium diffusion, sensory nerve stimulation, and tissue injury to occur.

Two models were used. First, a LMW fraction was isolated from urine, and its cytotoxicity determined to cultured urothelial cells, and tested for potential neutralization of the toxic effects by prior incubation with the anionic PPS. The urinary toxic factor was just as capable of injuring the urothelium as PS and exposure of the toxic fraction to PPS neutralized the toxic effect (Table 7).

Second, a rodent urodynamics model⁴ was used in which the bladders of healthy rats were exposed to toxic factor or PS, both of which caused significant changes in urodynamic parameters (NVC) relative to potassium. The key point in this model is that an intact epithelium prevents potassium diffusion and secondary muscular contractions; for the muscle to react with spasms (FIG. 2B), potassium must diffuse through the epithelium. The data obtained using this in vivo model indicate physiologic epithelial damage and muscle reactions in the intact bladder, and help corroborate and substantiate the findings from the in vitro cytotoxicity model. Together, these models will be valuable in screening for toxic factors (cytotoxicity) and then demonstrating they are active in the intact bladder.

In the urodynamics model,⁴ KCl is infused before and after urothelial injury with PS and a marked increase in muscle reactivity is observed after the PS treatment. A rat with a healthy urothelium should be no more reactive to KCl than to Na+, as demonstrated herein. When the urothelium was injured with toxic factor or PS as a positive control, a marked increase in NVC was found due to altered urothelial permeability to KCl and rapid secondary muscular contraction. In addition, we found PPS blocked the NVC induced by toxic factor (Table 8).

### CONCLUSIONS

As shown herein, normal human urine contains LMW cations that are capable of injuring the bladder mucosa in vivo, resulting in increased epithelial permeability as indicated by potassium sensitivity. Further, the data indicate that PPS can neutralize the dialyzed toxic factor from urine. These findings suggest that PPS may operate to address pathophysiologic processes of IC (IC/BPS) both in the epithelium and in urine, assisting in repair/replacement of the epithelium and binding potentially injurious cations in the urine. These data also suggest that IC (IC/BPS) may be initiated by these naturally occurring urinary cations if not sequestered by other compounds.

### Example 5

Interstitial cystitis (IC) can be diagnosed by measuring the reduction in sialic acid content that is seen in the Tamm-Horsfall protein of IC (IC/BPS) patients compared to normal, asymptomatic subjects. In addition to directly measuring the total sialic acid content of IC (IC/BPS) patients versus normal, which showed approximately 80% reduction of sialic acid content in the IC (IC/BPS) patient, this can be corroborated with MALDI-TOF mass spectrometry. Specifically, using the MALDI-TOF mass spectrometry to analyze the glycosylation chains of the Tamm-Horsfall protein, it was determined that there were significant differences between the IC (IC/BPS) patients and the normal, asymptomatic subjects. By releasing the intact sugar chains from the molecule using specific enzymes, intact polysaccharide chains were obtained and then using the mass spectrometry their structure and nature were determined.

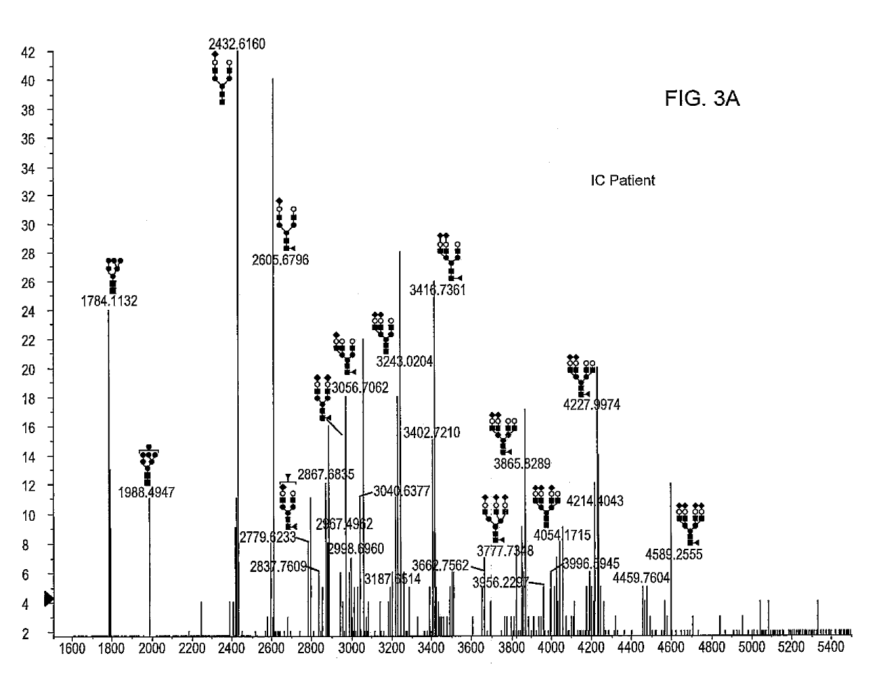

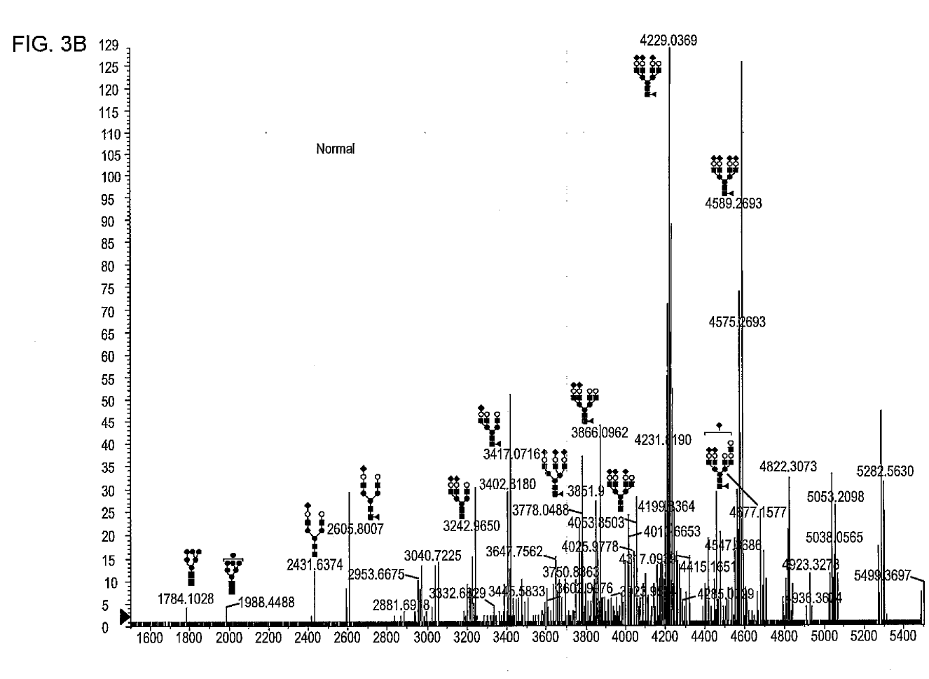

It was determined that in normal subjects, there were unusual heavy (>3800 daltons) polysaccharide chains that terminated in three and/or four sialic acids. These are called a “tri-antennary” or “tetra-antennary” structures, respectively. The normal subjects were rich in these particular polysaccharide chains (see FIGS. 3A and 3B). These structures results in three to four times as much sialic acid in the sugar chain compared to chains that terminate in zero, one or two sialic acids. On the other hand, IC (IC/BPS) patients showed a substantial reduction in the heavier polysaccharide chains which contain both tri-antennary and tetra-antennary chains, making them clearly separate and distinct from the normal asymptomatic subjects. In addition, it corroborated the fact that there was essentially almost three or four times as much sialic acid in the normal subjects compared to IC (IC/BPS) patients, because the “tri-antennary” and “tetra-antennary” chains have three to four sialic acids instead of zero, one or two sialic acids and these chains are much more abundant in the normal subject. This difference in the structure of these polysaccharide chains accounts for the approximately 3-4 fold increase that was seen in sialic acid in THP from normal people compared to IC (IC/BPS) patients.

As is noted in FIG. 3A, there is a marked difference in the presence of the chains with a heavier molecular weights (>3800 daltons) in that IC (IC/BPS) patients have a significant reduction in these heavy chains and these heavy chains represent the “tri-antennary” and “tetra-antennary” forms of the polysaccharides which end in three or four sialic acids. As shown in FIG. 3B, in the normal subject, a significantly greater amount of the heavier chains are present.

FIG. 4 depicts a normal subject showing the heavier chains with their specific molecular weights listed. In particular there is a large amount of these chains, e.g., note 4229.2068, 4589.3828 and 4459.1569 (as well as several others).

FIG. 5 depicts the polysaccharide chains in an IC (IC/BPS) patient. There is marked reduction in the heavy chains over 3800 molecular weight and the marked increase in the lighter chains such as seen in 2432 and 2605 (compared to the normal subjects). This reflects the significantly reduced “tri-antennary” and “tetra-antennary” polysaccharide chains in IC (IC/BPS) patients compared to normal people and accounts for the marked reduction in sialic acid content in the THP of IC (IC/BPS) patients compared to asymptomatic normal subjects.

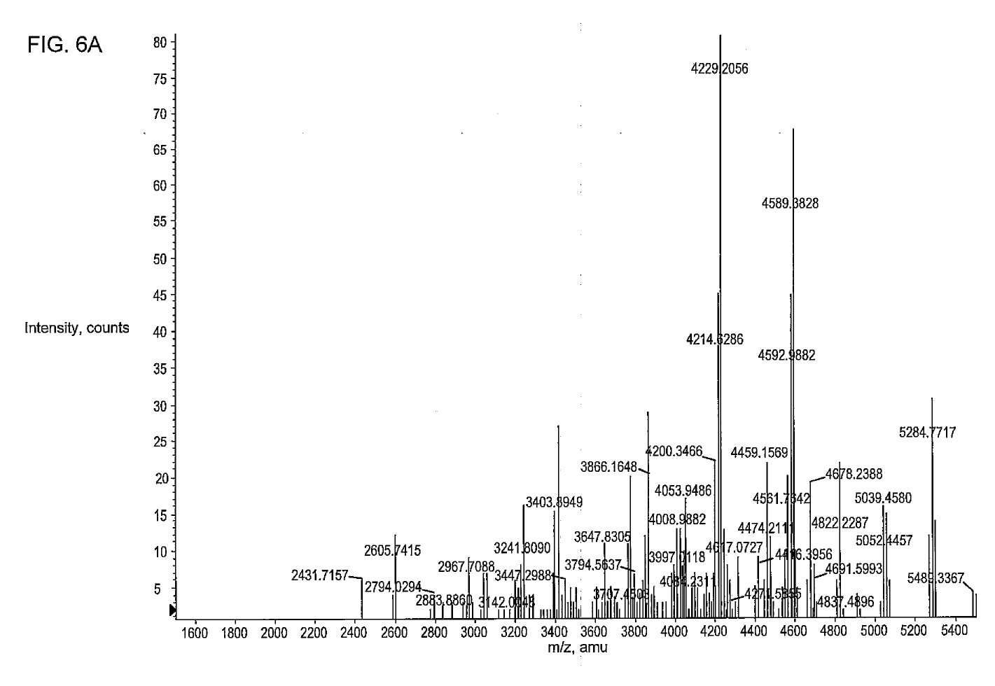

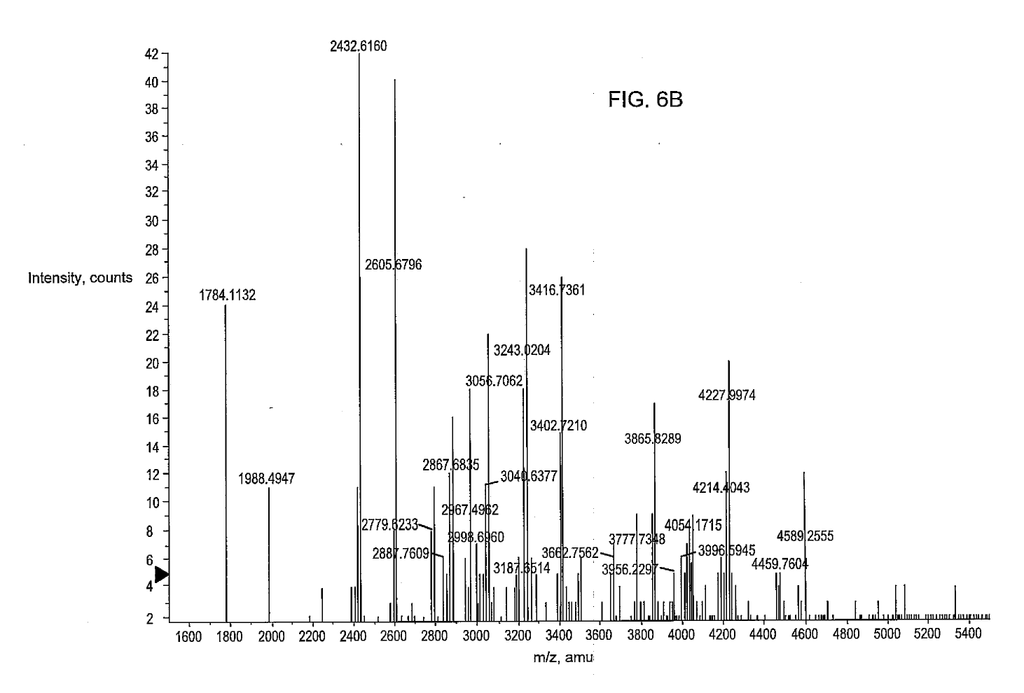

FIG. 6 depicts a side-by-side comparison of MALDI-TOF mass spectrometry of THP Glycosylation chains in control subjects (as shown in FIG. 4) and in IC (IC/BPS) patients (as shown in FIG. 5).

TABLE 9

| | High Levels of Tetra- | Low Levels of |
|---|---|---|
| Group | antennary Sialic Acid | Tetra-antennary Sialic Acid |
| Normals (10) | 10 | 0 |
| IC (IC/BPS)patients | 0 | 12* |
| (12) | | |

*p = <0.01

Table 9 is a summary showing the presence of high levels of tetra-antennary and terminal sialic acid on the polysaccharide chains of normals vs. the interstitial cystitics patients which shows a marked decrease in these polysaccharide chains that terminate in four sialic acids.

### Example 6

As discussed above, the ability of THP to neutralize the toxic factors could be affected by either a reduced concentration in urine of THP or a qualitative deficiency in the molecule itself. Applicant explored both possibilities.

### Materials and Methods

### Subjects

Urine samples were collected from female patients who were diagnosed with IC (IC/BPS) based on all of the National Institute of Diabetes and Digestive and Kidney Diseases clinical criteria excluding cystoscopy. In addition, patients must have had continuous symptoms for at least 12 months and a 24-hour micturition frequency of at least 12, and a PUF score of 15.⁹ Patients were a combination of new and treatment naïve as well as patients undergoing therapy.

Urine was collected from normal female subjects who had no history of any urinary bladder problems including infection, irritative voiding symptoms, or incontinence, no history of pelvic pain or vaginal problems such as vaginitis, vulvodynia or dyspareunia, and who scored 0 on the Pelvic Pain and Urgency/Frequency Patient Symptom Scale.⁹ The study was approved by the institutional review board and written informed consent was obtained prior to collection of all samples.

### Tamm Horsfall Protein Quantitation in Urine

For quantitation of Tamm Horsfall protein, fresh morning urine voids were collected in the Urology Clinic. These were processed by a filtration-washing protocol providing protein for further characterization by enzyme-linked immunosorbent assay (ELISA). Two 15-ml aliquots of urine were centrifuged sequentially until they resulted in a 30× concentration. The protein fraction greater than 30000 MW was washed by 2 centrifugations with 15 ml distilled water. The final material containing proteins greater than 30,000 daltons was brought up to 1 ml volume with water containing 0.01% azide preservative and stored at 4° C. until ELISA.

### ELISA for THP Quantitation

Protein isolated from urines was assayed by ELISA using 96-well plastic plates (Immulon®, Thermo Electron, Waltham, Mass.). Test plates were coated with purified THP (Biomedical Technologies, Inc., Stoughton, Mass.) 100 ng/well by incubating overnight in 0.05 M carbonate coating buffer (pH 9.6) at 4° C. and then washing twice with PBS and blocked in PBS+0.5% BSA-0.01% Tween-20 for 1 hr at room temperature. Wells were washed in PBS-0.01% Tween-20 (assay buffer, pH 7.4) and used immediately after washing with distilled water or stored after being dried. For quantitation of THP by the indirect ELISA, a standard curve was prepared by adding 100 μl of twofold serial dilutions of a THP standard (2.000-0.015 ng/well) to duplicate wells and immediately adding 100 μl of a 1/2000 dilution of goat anti-THP (ICN Pharmaceuticals, Inc., Costa Mesa, Calif.) to each well. A THP control, no THP (buffer alone), was prepared as above and mixed with the antibody. Samples were assayed in duplicate, 10 μl of each sample was added to 90 μl of assay buffer and 100 μl of the goat anti-THP added (1:2000) and the plate(s) incubated overnight on a shaker at room temperature. The assay plate was washed three times with assay buffer, then 100 μl of second antibody, rabbit anti goat-peroxidase (Sigma Chemical Co., St. Louis, Mo.) in assay buffer at 1:1000 dilution added for 1 hr. The plates were washed, and OPD peroxidase substrate (Sigma Chemical Co.) added for exactly 10 min in the dark, and the reaction stopped with dilute HCl. The plates were blanked to substrate in the plate reader and read at 450 nm. Average amounts of THP in the duplicate samples were extrapolated from the standard curve and the concentrations recorded as mg THP/L urine. THP was also normalized to urinary creatinine. Student's t test was used to determine significant differences between normal subjects and patients.

### Isolation of Tamm Horsfall Protein for N-glycan MALDI-MS Analysis and Quantitation of Sialic Acid

THP was isolated from subjects' urine via a salt precipitation method of Tamm and Horsfall.⁶ A total of 500 ml of fresh urine from each subject was utilized. Briefly, urine first underwent centrifugation to remove crystalloid and other debris. It was next incubated in the presence of 0.58M NaCl overnight at 4° C. followed by centrifugation. The THP pellet was rinsed twice with 0.58 M NaCl and resuspended in dIH₂O. This protein was then desalted by ultrafiltration with 1.5 L of dI H₂O (KrosFlo Research System II, Spectrum Labs, Rancho Dominguez, Calif.) utilizing a 50 kilodalton molecular weight cutoff filter (M15S-260-01N, Spectrum Labs). Desalted protein was lyophilized and stored dry at −70° C. Purity of the resulting THP was verified by SDS-PAGE electrophoresis followed by THP western blot. The quantity of protein used in all assays was determined by weighing lyophilized THP on a Mettler Toledo XS105 balance (accurate to 0.01 mg).

### Release of N-Glycans

Purified glycoproteins were treated with PNGase F in 20 mM HEPES buffer, pH 8.2 at 37° C. for 24 hr. The mixture was applied to a Sep-Pak C₁₈ cartridge equilibrated in water. Proteins and majority of the detergents bind to the resin. The run through and water washes were applied to a porous graphitized carbon (PGC) cartridge. Under aqueous conditions, oligosaccharides bind to the cartridge, while salts and buffers pass through unretarded. Sep-Pak C₁₈ and PGC cartridges were separately preconditioned using either methanol or acetonitrile. N-linked glycans were eluted with 30% acetonitrile containing 0.1% TFA. N-glycan fraction was dried by speed-vac and ready for further analysis.

### Permethylation of N-Glycans

N-glycans were dried under vacuum desiccators overnight and permethylated using slurry made NaOH in anhydrous DMSO. Iodomethane was added and the mixture stirred vigorously for 1 hr at room temperature. The reaction was quenched by slow dropwise addition of 1% acetic acid, chloroform and water were added, mixed thoroughly and allowed to settle into two layers. The aqueous layer was removed and discarded and the chloroform layer dried down under a stream of nitrogen. The dried mixture was redissolved in 50% methanol and loaded into Sep-Pak cartridge for stepwise elution with water, and then with 15%, 35%, 50% and 75% aqueous acetonitrile. Each acetonitrile fraction was collected and evaporated to dryness. The samples were now ready for mass spectrometry (MS) analysis.

### MALDI-TOF MS Analysis of Permethylated Glycans

MALDI-TOF MS analysis was performed in positive reflectron mode using ABI QSTAR XL MALDI-TOF (Applied Biosystems, Foster City, Calif.). Derivatized permethylated glycans were dissolved in methanol/water (8:2) and mixed in 1:1 ratio with 10 mg/ml 2,5-dihydroxybenzoic acid in 80:20 methanol:water. 1.5 μl aliquots were spotted onto a 100-well sample plate and dried under air. Permethylated maltose was used for external calibration. Ten control subjects and ten IC (IC/BPS) patients were randomly selected for analysis as confirmation of the actual sialic acid measurements via a different technology.

### DMB-HPLC Analysis of Sialic Acids

Samples were heated to 80° C. in 2 M acetic acid for 3 hr. The released sialic acids were collected by ultrafiltration through a 3000 MWCO filter and derivatized with 1,2-diamino-4,5-methylenedioxybenzene (DMB) (Sigma Aldrich, Saint Louis, Mo.) as described in Hara et al.⁴⁷ The fluorescent, DMB-derivatized sialic acids were analyzed by reversed-phase high-performance liquid chromatography (HPLC) using an Acclaim 120 C₁₈ column (Dionex) at a flow rate of 0.9 mL/min. Samples were eluted with a gradient of acetonitrile (8-11%) in methanol (7%) and H₂O over 39 min followed by 11 min at the final conditions. The excitation and emission wavelengths were 373 and 448 nm, respectively. The DMB-derivatized sialic acids were identified and quantified by comparing elution times and peak areas to known standards that were similarly treated.

### Monosaccharide Analysis of Neutral and Amino Sugars

THP was isolated from urine samples of IC/BPS patients and control subjects by salt precipitation. The purified THP was hydrolyzed in 2M Trifluoroacetic acid at 100° C. for 4 h to release monosaccharides. After drying the hydrolyzate, samples were dissolved in water and analyzed with high pH anion exchange chromatography with pulsed amperometric detection (HPAEC-PAD). Neutral and amino sugars were separated on CarboPac PA 10 column (4×250 mm) with AminoTrap guard column (4×50 mm) using isocratic gradient of 18 mM sodium hydroxide at flow rate of 1 ml/min. Data was collected using Dionex DX-600 HPLC system. Identification and quantification of all sugars was obtained by comparison with the standards.

### THP 2-AB N-Glycan Profiling

THP was isolated from urine samples of IC/PBS patients and control subjects by salt precipitation. The purified THP was treated with PNGaseF to release N-glycans. The N-glycans were purified using Sep-Pak C₁₈ cartridges followed by porous graphitized carbon cartridges. The purified N-glycans were labeled with 2-Aminobenzamide (2-AB). The 2-AB labeled glycans were cleaned up using Glyco-Clean S cartridges (GLYKO). HPLC profiling of 2-AB derivatized N-glycans was obtained by using CarboPac PA1 anion exchange column in 100 mM sodium hydroxide with 0-250 mM sodium acetate gradient. Data was collected using UltiMate 3000 (Dionex) HPLC system with RF 2000 fluorescence detector set at λₑₓ 330 nm and λₑₘ 420 nm.

### Statistics

For comparison of both the quantity of THP in normal subjects versus IC (IC/BPS) patients and the difference in sialic acid content, the Student's t test was employed. To compare the results on the MALDI-TOF MS, chi-square analysis was used because there were only two groups of THP chains seen, those with a preponderance of heavy polysaccharide chains and those with a preponderance of light polysaccharide.

### Results

The IC (IC/BPS) subjects had a median age of 41 years and the control subjects, 38 years.

The absolute amount of THP in urine was not significantly different in IC (IC/BPS) patients versus healthy control subjects (28.8 versus 28.2 mg/L urine and 36.8 versus 36.7 μg/mg creatinine, respectively) (Table 10).

TABLE 10
Total THP in IC vs normal urine.

Urinary THP

| | mg/l Urine | μg/mg Creatinine |
|---|---|---|
| No. pts: | | |
| IC | 115 | 35 |
| Normal | 39 | 25 |
| Mean ± SD: | | |
| IC | 28.8 ± 19.1 | 36.8 ± 21.2 |
| Normal | 28.2 ± 20.8 | 36.7 ± 23.1 |
| p Value | Not Significant | 0.596 |

THP concentrations were the same in patients with IC (IC/BPS) controls with protein harvested from fresh urine samples by ultrafiltration to avoid any protein loss.
*Concentrations of THP were the same in IC (IC/BPS) patients and controls. The protein was harvested from fresh urine samples by ultrafiltration to avoid any loss of protein.

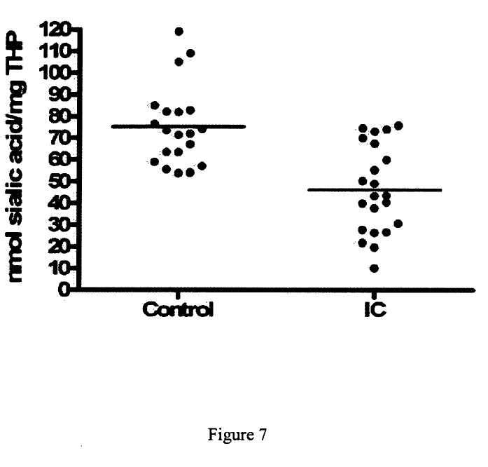

Total sialic acid was almost twofold lower in 22 patients with IC (IC/BPS) than in 20 controls (mean±SEM 46.3±4.3 versus 75.3±4.1 nanomoles sialic acid/mg THP, respectively; p<0.0001) (FIG. 7).

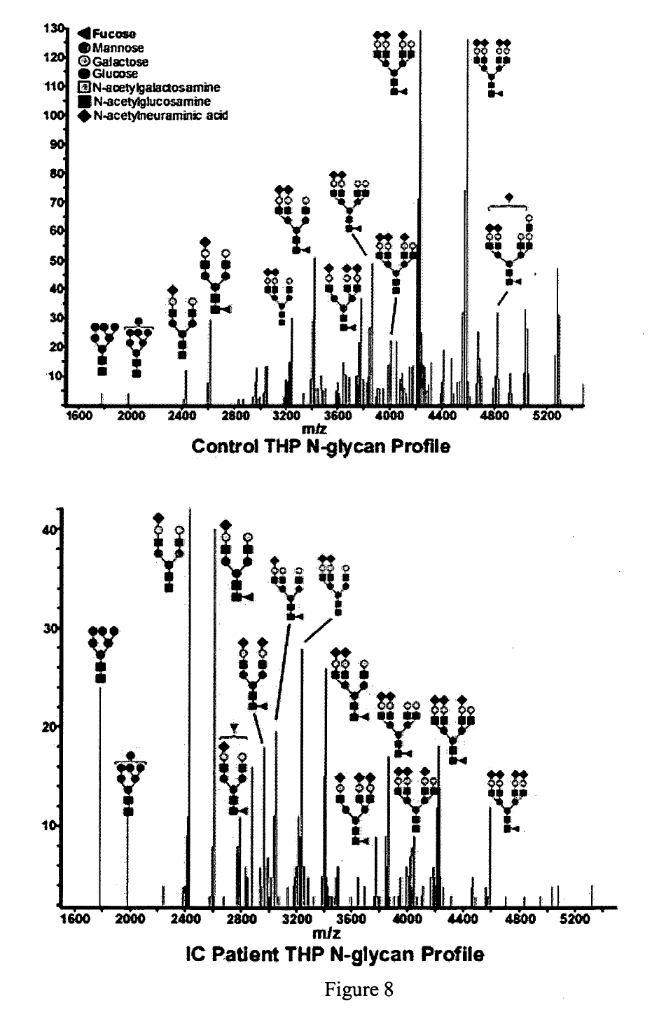

MALDI-TOF MS on N-glycans released from THP of healthy control subjects (n=10) revealed that the pattern of N-glycosylation is qualitatively different in all controls versus all IC (IC/BPS) patients (n=10) (FIGS. 8A and 8B). IC (IC/BPS) patients (FIG. 8B) display mass spectra with lower molecular weight di-antennary N-glycan structures, with a resulting reduction in the number of terminal sialic acid residues. In IC (IC/BPS) patients versus normal subjects, there was a highly significant difference in frequency of having a preponderance of heavy chains versus light chains (p<0.0001).

### 2AB Derivative Analysis of THP Glycosylation Chains Using High Performance Liquid Chromatography (HPLC)

Using HPLC we determined the ratio of light weight glycosylation chains to heavy weight chains (that contain the sialic acid) and found a deficiency of heavy weight (sialylated chains) in 10 IC patients compared to 10 normal healthy control subjects. The average ratio for the IC patients was 3.2 and for the control subjects was 2.1, indicating that the IC patients are deficient in heavy weight glycosylation chains that contain the sialic acid compared to control subjects.

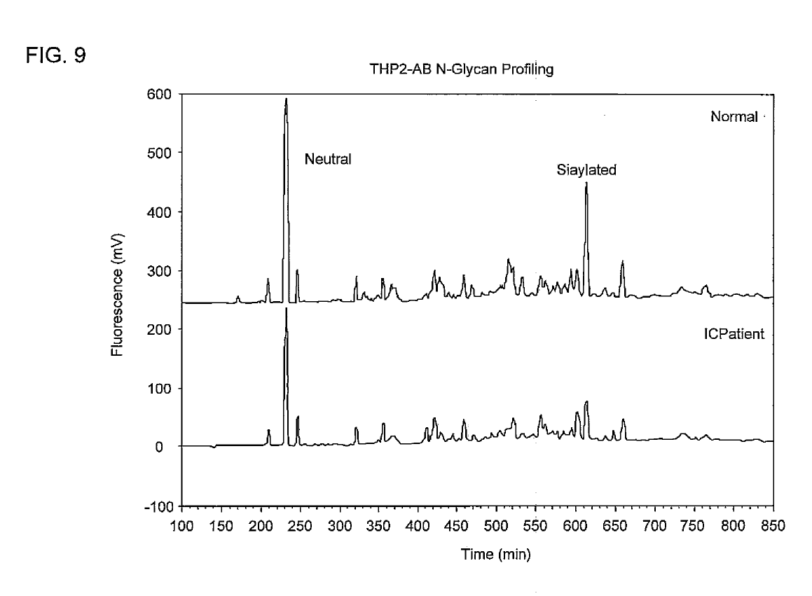

FIG. 9 shows an HPLC profile of 2-AB labeled N-Glycans of normal versus. IC patient. The pattern of reduced level of high molecular weight sialylated oligosaccharide was seen in four out of 20 normal subjects versus in 15 out of 20° C. patients and this difference was significant p<0.0001.

### Carbohydrate (for Example, Monosaccharide) Content of THP in IC/BPS Patients and Normal Healthy Control Subjects:

The 2 AB profiling of N-Glycan suggested that many of the IC patients not only had lower sialic acid content but significantly reduced glycosylation overall. To confirm this observation and potentially increase our sensitivity and specificity of detecting THP abnormalities in IC/BPS patients we measured the total monosaccharide content THP in a normal group and IC/BPS patients.

The monosaccharide concentration of 10 normal subjects averaged 137 nM compared to 97 nM in 10 IC (IC/BPS) patients. These results reflect the overall reduction of glycosylation in patients with Interstitial Cystitis (IC/BPS) and reflect another method to determine it. The results are summarized in Table 11. As used herein, “FUC” is fucose, “GalNAc” is N-acetylgalactosamine, “GlcNAc” is N-acetylglucosamine, “Gal” is Galactose and “Man” is Mannose.

TABLE 11
Monosaccharide analysis of THP; 100ul (200ug THP was analyzed)

| | | | | | | |
|---|---|---|---|---|---|---|
| Sample | FUC | GalNAc | GlcNAc | Gal | Man | Total |

Normals
nM in 100% sample

| | | | | | | |
|---|---|---|---|---|---|---|
| JP 201 | 6.45 | 17.5 | 37.8 | 26.9 | 18.8 | 107.45 |
| JP 203 | 8.2 | 9.6 | 60 | 46 | 39.8 | 163.6 |
| JP 204 | 8.4 | 12 | 48.8 | 37.5 | 32 | 138.7 |
| N1 | 7.95 | 3.05 | 53.9 | 41.1 | 35.4 | 141.4 |
| N12 | 7.5 | 6.1 | 37.8 | 29.3 | 22.2 | 102.9 |
| N2 | 9.3 | 8.8 | 63.3 | 50 | 43.6 | 175 |
| N23 | 6.85 | 10.3 | 46.4 | 35.5 | 30.4 | 129.45 |
| N27 | 9 | 13.1 | 56.4 | 42.8 | 37.4 | 158.7 |
| N28 | 9 | 8.35 | 46.9 | 36.1 | 30.3 | 130.65 |
| N4 | 8.55 | 5.1 | 46.8 | 36 | 26.9 | 123.35 |
| Average | 8.12 | 9.39 | 49.81 | 38.12 | 31.68 | 137.12 |

Interstitial Cystitis

| | | | | | | |
|---|---|---|---|---|---|---|
| ICF 31 | 4.6 | 9.45 | 22.6 | 21.6 | 27.9 | 86.15 |
| ICF 37 | 5.95 | 8.6 | 37.8 | 41.6 | 36.1 | 130.05 |
| ICF 40 | 3.6 | 3.75 | 25.1 | 19.2 | 18.8 | 70.45 |
| ICF 41 | 3.55 | 6.75 | 24 | 19.3 | 12 | 65.6 |
| ICF 94 | 8.15 | 10.7 | 42 | 30.4 | 26 | 117.25 |
| ICF 96 | 8.8 | 4.45 | 28.3 | 20.3 | 28.8 | 90.65 |
| ICF 99 | 5.75 | 3.1 | 23.7 | 25.6 | 12.4 | 70.55 |
| JP 202 | 11.1 | 6.85 | 44.3 | 30.9 | 26.4 | 119.55 |
| JP 206 | 11.8 | 14.6 | 53.3 | 36.2 | 31.6 | 147.5 |
| JP 208 | 7.25 | 6.45 | 25.8 | 19.4 | 14.8 | 73.7 |
| Average | 7.06 | 7.47 | 32.69 | 26.45 | 23.48 | 97.145 |

### DISCUSSION

IC (IC/BPS) patients and controls had nearly identical urinary levels of THP. However, THP from IC (IC/BPS) patients exhibited nearly threefold less total sialic acid per milligram protein and a qualitatively different pattern of N-glycosylation relative to normal controls.

Together with the evidence that healthy individuals' THP protects the bladder epithelium from injury by known cytotoxic agents^{7, 23} and that THP from IC (IC/BPS) patients is less protective against bladder epithelial injury than THP from healthy individuals,⁷ these data raise the possibility that a structural alteration in this protein may figure prominently in the pathogenesis of IC.

There is extensive evidence that the pathogenesis of IC (IC/BPS) involves a functional deficit in the glycosaminoglycan-rich mucus layer that insulates the transitional epithelium of the bladder from toxic urinary solutes.^{1, 13, 14, 23, 39, 40, 46} Several in vitro studies in urothelial cell model systems have shown that low molecular weight (500-1000 Daltons) cationic toxic urinary solutes, or toxic factors, exert significant cytotoxic activity.^{4, 23} In animal models, exposure of the bladder mucus layer to toxic factors and protamine results in loss of epithelial barrier function.^{1, 8, 23, 40} In turn, loss of the epithelial barrier allows the urinary solute, potassium, to move into the bladder interstitium and provoke contractions of bladder smooth muscle.^{1, 8, 23, 28, 40, 48} These findings illustrate the mechanism we have proposed for the provocation of frequency, urgency, pain, incontinence, and eventual bladder tissue damage as a result of a dysfunctional bladder mucus layer in IC (IC/BPS) patients.

The possibility that such a cascade of events can lead to disease has raised the question of whether the urine contains compounds whose function is to offset or neutralize the toxic factors. In in vitro studies, we have found that normal subjects' THP significantly reduces the toxicity of two cationic substances, toxic factor and protamine sulfate.^{4, 7, 23} Protamine sulfate, employed as a positive control in these studies, contains highly charged quaternary amines that are well known to destroy the protective mucus barrier.^{1, 7, 13, 40} Further, normal subjects' THP is significantly more protective against cations than the protein from IC (IC/BPS) patients.⁷ When normal subjects' THP is desialylated, its protective effect decreases significantly.⁴⁶ These findings suggest that THP inactivates the urinary toxic factors, possibly by binding to them in a manner analogous to that of albumen in blood. Compounds bound to serum proteins are biologically inactive. THP, with its anionic charge, could bind to the cationic toxic factors via electrostatic interaction.

In the current study, the nearly threefold difference in THP sialic acid content between IC (IC/BPS) patients and controls on HPLC-based assay was substantiated by the results of MALDI-TOF mass spectrometry on the protein's intact glycosylation chains. The mass spectra of normal THP N-glycans display a preponderance of heavy chains over 3800 daltons (FIG. 8A). These heavy chains are known to end as tri- and tetra-antennary structures that contain at the N-terminal glycans either 3 or 4 sialic acid residues.^{42, 43} Mass spectra of an IC (IC/BPS) patient's THP (FIG. 8B) reveal most of the carbohydrate chains to be of lower molecular weight. These lighter chains are known to have 0, 1 or 2 sialic acid moieties at the terminus of the N-glycan chain.^{42, 43}

With most of the control subjects' THP carbohydrate chains ending in 3-4 sialic acids and the patients' in 0-2 sialic acids, one would expect that the sialic acid content in control subjects' THP would be approximately 3 times greater than that in IC (IC/BPS) patients. The data from our HPLC-based sialic acid assay demonstrates that this is the case.

As a composite, these data have major implications for explaining the function of THP as well as for the understanding of the causes and treatment of IC. First, these are the first substantial data to propose a major function for THP in the urinary tract, namely, as a protective urinary macromolecule that functions by biologically inactivating cations that have the potential of injuring mucus. Because such protective activity would prevent the subsequent induction of IC-associated symptoms and pathology, defective THP could be the primary factor in the cause of IC.

These data suggest that THP is a novel diagnostic marker for IC (IC/BPS) with superior sensitivity and specificity over that of currently available diagnostic tools. Additionally, if a defective protein initiates the IC (IC/BPS) cascade, then genetics is implicated in IC.

### REFERENCES

1. Parsons C L, Greenberger M, Gabal L, Bidair M, Barme, G. J Urol 1998; 159: 1862-1867.
2. Parsons C L, Stein P C, Bidair M, Lebow D. Neurourol Urodyn 1994; 13: 515-520.
3. Parsons C L. Urology 2003; 62: 976-982.
4. Parsons C L, Bautista S L, Stein P C, Zupkas P. J Urol 2000; 164: 1381-1384.
5. Badgett A, Kumar S. Urol Int 1998; 61: 72-75.
6. Tamm I, Horsfall F L. Proc Soc Exp Biol Med 1950; 74: 108-114.
7. Akiyama A, Stein P C, Houshiar A, Parsons C L. Internat J Urol 2000; 7: 176-183.
8. Chuang Y C, Chancellor M B, Seki S, Yoshimura N, Tyagi P, Huang L, et al. Urology 2003; 61: 664-670.
9. Parsons C L, Dell J, Stanford E J, Bullen M, Kahn B S, Waxell T, et al. Urology 2002; 60: 573-578.
10. Lilly J D, Parsons C L. Surg Gynec Obst 1990; 171: 493-496.
11. Parsons C L, Stauffer C, Schmidt J D. Science 1980; 208: 605-607.
12. Parsons C L, Boychuk D, Jones S, Hurst R, and Callahan H. J Urol 1990; 143: 139-142.
13. Parsons C L, Lilly J D, Stein P C. J Urol 1991; 145: 732-735.
14. Buffington C A T, Woodworth B E. J Urol 1997; 158: 786-789.
15. Chelsky M J, Rosen S I, Knight L C, Maurer A H, Hanno P M, Ruggieri M R. J Urol 151: 346-349, 1994.
16. Erickson D R, Herb N, Ordille S, Harmon N, Bhavanandan V P. J Urol 164: 419-422, 2000.
17. Hohlbrugger G. Sant G R (Ed): Interstitial Cystitis. Philadelphia, Lippincott-Raven Publishers, 1997, pp. 87-92.
18. Parsons C L, Greene R A, Chung M, Stanford E J, Singh G. J Urol 2005; 173:1182-1185.
19. Daha L K, Riedl C R, Lazar D, Hohlbrugger G, Pfluger H. Eur Urol 2005; 47:393-397.
20. Grégoire M, Liandier F, and Naud A, Lacombe L, Fradet Y. J Urol 2002; 168: 556-557.
21. Chung M K, Chung R P, Gordon D. JSLS 2005; 9:25-9.
22. Keay S K, Szekely Z, Conrads T P, Veenstra T D, Barchi J J Jr, Zhang C O, et al. Proc Natl Acad Sci USA. 2004; 101:11803-8.
23. Stein P, Rajasekaran M, Parsons C L: Urology 66: 903-907, 2005.
24. Easton R L, Patankar M S, Clark G F, et al: J Biol Chem 275: 21928-21938, 2000.
25. R. E. Hurst, R. Zebrowski, J. Urol. 152, 1641 (1994).
26. J. Cornish, J. C. Nickel, M. Vanderwee, J. W. Costerton, Urol. Res. 18, 263 (1990).
27. N. G. Moss, W. W. Harrington, M. S. Tucker, Am. J. Physiol. 272, R695 (1997).
28. L. K. Daha et al., J. Urol. 170, 807 (2003).
29. M. Rajasekaran, P. Stein, C. L. Parsons, Int. J. Urol. 13, 409 (2006).
30. C. C. Gryte, H. P. Gregor, J. Polymer Sci. 14, 1839 (1976).
31. H. P. Gregor, in Charged Gels and Membranes, Part I, E. Sélégny, Ed. (D. Reidel, Boston, 1976), pp. 235-254.
32. H. P. Gregor, in Biomedical Applications of Polymers, H. P. Gregor, Ed. (Plenum, New York, 2005), pp. 51-56.
33. J. M. Dietschy, V. L. Sallee, F. A. Wilson, Gastroenterology 61, 932 (1971).
34. F. A. Wilson, V. L. Sallee, J. M. Dietschy, Science 174, 1031 (1971).
35. H. Westergaard, J. M. Dietschy, J. Clin. Invest. 58, 97 (1976).
36. Shirley S W, Stewart B H, Mirelman S., Urology, 11, 215-20 (1978)
37. Parsons, C L, Housley T, Schmidt J D, Lebow D, Br J Urol, 73, 504-7 (1994)
38. Parsons, C L, Urology, 65, 45-8 (2005)
39. Hurst, R. E.: Structure, function, and pathology of proteoglycans and glycosaminoglycans in the urinary tract. World J Urol, 12: 3, 1994
40. Parsons, C. L.: The role of the urinary epithelium in the pathogenesis of interstitial cystitis/prostatitis/urethritis. Urology, 69: S9, 2007
41. Muchmore, A. V., Decker, J. M.: Uromodulin: a unique 85-kilodalton immunosuppressive glycoprotein isolated from urine of pregnant women. Science, 229: 479, 1985
42. van Rooijen, J. J., Voskamp, A. F., Kamerling, J. P., Vliegenthart, J. F.: Glycosylation sites and site-specific glycosylation in human Tamm-Horsfall glycoprotein. Glycobiology, 9: 21, 1999
43. van Rooijen, J. J., Kamerling, J. P., Vliegenthart, J. F.: Sulfated di-, tri- and tetraantennary N-glycans in human Tamm-Horsfall glycoprotein. Eur J Biochem, 256: 471, 1998
44. Bates, J. M., Raffi, H. M., Prasadan, K., Mascarenhas, R., Laszik, Z., Maeda, N. et al.: Tamm-Horsfall protein knockout mice are more prone to urinary tract infection: rapid communication. Kidney Int, 65: 791, 2004
45. Hallson, P. C., Choong, S. K., Kasidas, G. P., Samuell, C. T.: Effects of Tamm-Horsfall protein with normal and reduced sialic acid content upon the crystallization of calcium phosphate and calcium oxalate in human urine. Br J Urol, 80: 533, 1997
46. Parsons, C. L., Rajasekaran, M., Arsanjani, A. H., Chenoweth, M., Stein, P.: Role of sialic acid in urinary cytoprotective activity of Tamm-Horsfall protein. Urology, 69: 577, 2007
47. Hara, S., Takemori, Y., Yamaguchi, M., Nakamura, M., Ohkura, Y.: Fluorometric high-performance liquid chromatography of N-acetyl- and N-glycolylneuraminic acids and its application to their microdetermination in human and animal sera, glycoproteins, and glycolipids. Anal Biochem, 164: 138, 1987
48. Philip, J., Willmott, S., Irwin, P.: Interstitial cystitis versus detrusor overactivity: a comparative, randomized, controlled study of cystometry using saline and 0.3 M potassium chloride. J Urol, 175: 566, 2006

What is claimed:

1. A method for inhibiting Interstitial Cystitis and its symptoms in a subject comprising administering an effective amount of a Tamm-Horsfall protein to the subject, so as to inhibit Interstitial Cystitis and its symptoms in the subject.

2. A method for reducing symptoms of Interstitial Cystitis in a subject by inhibiting Interstitial Cystitis by the method of claim 1, thereby reducing the symptoms of Interstitial Cystitis.

3. A method for increasing the levels of Tamm-Horsfall protein in a subject comprising administering to the subject an effective amount to Tamm-Horsfall protein, thereby increasing the levels of Tamm-Horsfall protein.

4. A method for repairing a mucin layer of bladder in a subject by increasing the levels of Tamm-Horsfall protein by the method of claim 3, so as to repair the mucin layer of the bladder.

5. A method for treating a disease associated with decreased levels of Tamm-Horsfall protein in a subject by increasing the levels of Tamm-Horsfall protein by the method of claim 3, thereby treating the disease.

6. A method for diagnosing Interstitial Cystitis in a subject comprising quantitatively determining in the urine from the subject, the levels of Tamm-Horsfall protein and comparing the amount of Tamm-Horsfall protein so determined to the amount in a sample from a normal subject, the decrease in the amount of Tamm-Horsfall protein being indicative of Interstitial Cystitis.

7. A method for monitoring the course of Interstitial Cystitis in a subject which comprises quantitatively determining in a first sample of a urine from the subject the levels of Tamm-Horsfall protein and comparing the amount so determined with the amount present in a second sample from the subject, such samples being taken at different points in time, a difference in the levels of Tamm-Horsfall protein determined being indicative of the course of Interstitial Cystitis.

8. The method of claims 1, 2, 3, 4, or 5, wherein the Tamm-Horsfall protein is administered directly in to the urinary tract in a subject.

9. The method of claim 8, wherein the Tamm-Horsfall protein is administered using a catheter.

10. The method of claim 8, wherein the Tamm-Horsfall protein is administered using a time-release system.

11. The method of claim 10, wherein the time-release system is a balloon catheter.

12. The method of claims 1, 2, 3, 4, 5, 6, or 7, wherein the Tamm-Horsfall protein is sialylated.

13. A method for screening for agents that modulate production of Tamm-Horsfall protein comprising:
a) contacting Tamm-Horsfall genes in Tamm-Horsfall positive cells with a molecule of interest; and
b) determining whether the contact results in increased Tamm-Horsfall production, increased Tamm-Horsfall production being indicative that the molecule modulates production of Tamm-Horsfall genes.

14. A method for screening for agents that modulate production of Tamm-Horsfall protein comprising:
a) contacting Tamm-Horsfall protein in Tamm-Horsfall positive cells with a molecule of interest; and
b) determining whether the contact results in increased Tamm-Horsfall production, increased Tamm-Horsfall production being indicative that the molecule modulates production of Tamm-Horsfall protein.

15. The method of claims 13 or 14, wherein the agent that modulates production of Tamm-Horsfall protein is a reproductive hormone.

16. The method of claim 15, wherein the hormone is estrogen.

17. The method of claim 15, wherein the hormone is progesterone.

18. A method for screening for agents that modulate sialylation of Tamm-Horsfall protein comprising:
a) contacting Tamm-Horsfall protein in Tamm-Horsfall positive cells with a molecule of interest; and
b) determining whether the contact results in increased sialylation of Tamm-Horsfall protein, increased sialylation of Tamm-Horsfall protein being indicative of modulation of sialylation of Tamm-Horsfall protein.

19. The method of claim 18, wherein increased sialylation of Tamm-Horsfall protein is measured by measuring the zeta-potential of the Tamm-Horsfall protein.

20. The method of claims 13, 14 or 18, wherein the agent is a small molecule, protein, peptide, or a combination thereof.

21. A screening method according to claims 13, 14 or 18, which comprises separately contacting each of a plurality of samples to be tested.

22. The screening method of claim 21, wherein the plurality of samples comprises more than about 104 samples.

23. The screening method of claim 21, wherein the plurality of samples comprises more than about 5×10⁴ samples.

24. The method of claims 1, 2, 3, 4, 6 or 7, wherein the subject is selected from the group consisting of human, monkey, ape, dog, cat, cow, horse, rabbit, mouse and rat subjects.

25. A pharmaceutical composition comprising Tamm Horsfall protein and a pharmaceutically acceptable carrier.

26. The pharmaceutical composition of claim 25, wherein the Tamm-Horsfall protein is sialylated.

27. A kit comprising the pharmaceutical composition of claims 25 or 26.

28. A method for diagnosing Interstitial Cystitis in a subject comprising quantitatively determining in the urine from the subject, the amount of sialylation of Tamm-Horsfall protein and comparing the amount of sialylation of Tamm-Horsfall protein so determined to the amount in a sample from a normal subject, the decrease in the amount of sialylation of Tamm-Horsfall protein being indicative of Interstitial Cystitis.

29. A method for monitoring the course of Interstitial Cystitis in a subject which comprises quantitatively determining in a first sample of a urine from the subject the amount of sialylation of Tamm-Horsfall protein and comparing the amount so determined with the amount present in a second sample from the subject, such samples being taken at different points in time, a difference in the levels of sialylation of Tamm-Horsfall protein determined being indicative of the course of Interstitial Cystitis.

30. A method for diagnosing Interstitial Cystitis in a subject comprising quantitatively determining in the urine from the subject, the total amount of carbohydrates in Tamm-Horsfall protein and comparing the amount of carbohydrates in Tamm-Horsfall protein so determined to the amount in a sample from a normal subject, a decrease in the total amount of carbohydrates in Tamm-Horsfall protein being indicative of Interstitial Cystitis.

31. A method for monitoring the course of Interstitial Cystitis in a subject which comprises quantitatively determining in a first sample of a urine from the subject the total amount of carbohydrates in Tamm-Horsfall protein and comparing the amount so determined with the amount present in a second sample from the subject, such samples being taken at different points in time, a difference in the total amount of carbohydrates in Tamm-Horsfall protein determined being indicative of the course of Interstitial Cystitis.

32. The method of claim 6, further comprising determining in the urine from the subject, the amount of sialylation of Tamm-Horsfall protein and comparing the amount of sialylation of Tamm-Horsfall protein so determined to the amount in a sample from a normal subject, a decrease in the amount of sialyation of Tamm-Horsfall protein being indicative of Interstitial Cystitis.

33. The method of claim 6, further comprising determining in the urine from the subject, the amount of total carbohydrates in Tamm-Horsfall protein and comparing the amount of total carbohydrates in Tamm-Horsfall protein so determined to the amount in a sample from a normal subject, a decrease in the amount of total carbohydrates in Tamm-Horsfall protein being indicative of Interstitial Cystitis.

34. The method of claim 28 further comprising quantitatively determining in the urine from the subject, the amount of Tamm-Horsfall protein and comparing the amount of Tamm-Horsfall protein so determined to the amount in a sample from a normal subject, a decrease in the amount of Tamm-Horsfall protein being indicative of Interstitial Cystitis.

35. The method of claim 28 further comprising quantitatively determining in the urine from the subject, the amount of total carbohydrates in Tamm-Horsfall protein and comparing the amount of total carbohydrates) in Tamm-Horsfall protein so determined to the amount in a sample from a normal subject, a decrease in the amount of total carbohydrates in Tamm-Horsfall protein being indicative of Interstitial Cystitis.

36. The method of claim 30 further comprising quantitatively determining in the urine from the subject, the total amount of Tamm-Horsfall protein and comparing the amount of Tamm-Horsfall protein so determined to the amount in a sample from a normal subject, a decrease in the amount of Tamm-Horsfall protein being indicative of Interstitial Cystitis.

37. The method of claim 30 further comprising quantitatively determining in the urine from the subject, the amount of sialylation of Tamm-Horsfall protein and comparing the amount of sialylation of Tamm-Horsfall protein so determined to the amount in a sample from a normal subject, a decrease in the amount of sialylation of Tamm-Horsfall protein being indicative of Interstitial Cystitis.

38. The method of claim 37 further comprising quantitatively determining in the urine from the subject, the amount of Tamm-Horsfall protein and comparing the amount of Tamm-Horsfall protein so determined to the amount in a sample from a normal subject, the decrease in the amount of Tamm-Horsfall protein being indicative of Interstitial Cystitis.

39. The method of claims 30, 33 or 35, wherein the Tamm-Horsfall protein in Interstitial Cystitis subjects comprises reduced amounts of heavy weight glycosylation chains compared to normal subjects.

40. The method of claims 30, 33 or 35, wherein the carbohydrates in the Tamm-Horsfall protein are monosaccharides.
